(19) United States
(12) Patent Application Publication
(10) Pub. No.: US 2022/0057377 A1
(43) Pub. Date: Feb. 24, 2022

(54) ANALYTICAL METHOD FOR DETERMINATION OF IN VITRO DRUG RELEASE PROFILE FROM MICRONEEDLE PATCHES

(71) Applicant: ZOSANO PHARMA CORPORATION, Fremont, CA (US)

(72) Inventors: Aihua He, Sunnyvale, CA (US); Mahmoud Ameri, Fremont, CA (US); Hayley Lewis, Redwood City, CA (US)

(21) Appl. No.: 17/392,536

(22) Filed: Aug. 3, 2021

Related U.S. Application Data
(60) Provisional application No. 63/060,336, filed on Aug. 3, 2020.

(51) Int. Cl. G01N 33/15; G01N 13/00

(52) U.S. Cl. CPC G01N 33/15; G01N 2013/006; G01N 13/00

(57) ABSTRACT

The present disclosure relates to a novel in vitro dissolution method to assess the release of a bioactive agent from an intracutaneous microneedle system wherein the bioactive agent is coated on or integral to the microneedles. The present disclosure further relates to a novel sample holder clip assembly specially adapted for use with microneedle arrays and patches.

### RELATED APPLICATION

This application claims priority to U.S. Provisional Patent Application Ser. No. 63/060,336 filed on Aug. 3, 2020, the disclosure of which is hereby incorporated by reference to the full extent permitted by law.

### FIELD

The present disclosure relates to the field of in vitro dissolution methods, and more particularly to an in vitro dissolution method to assess the release of a bioactive agent from an intracutaneous microneedle system wherein the bioactive agent is coated on a metallic or polymeric substrate.

### BACKGROUND

Dissolution testing measures the extent and rate of solution formation from a dosage form, such as a tablet, a capsule, ointment, etc. The dissolution of a drug is important for its bioavailability and therapeutic effectiveness.

In vitro drug release is an important component of drug product characterization and is routinely used as a quality control test in assessing reproducibility of the drug manufacturing process. To properly evaluate the dissolution of drug products, it is critical to have standardized procedures. Standardized procedures help to ensure consistent quality in production and may, in some circumstances, serve as a predictive measure of the characteristics of a dosage form and drug under in vivo conditions.

Generally, a dissolution test uses an apparatus with specific test conditions in combination with acceptance criteria to evaluate the performance of the subject dosage form. There are a number of ways that dissolution has been tested. For example, the United States Pharmacopeia's general chapter, USP <711>, describes four types of apparatus. Apparatus 1, the basket apparatus, consists of the following: a vessel, which may be covered, made of glass or other inert, transparent material; a motor; a metallic shaft; and a cylindrical basket. The vessel is partially immersed in a suitable water bath or heated by a suitable device such as a heating jack. Apparatus 2, the paddle apparatus, uses the assembly from Apparatus 1, except that a paddle, formed from a blade and shaft, is used as the stirring element. Apparatus 3, the reciprocating cylinder, consists of a set of cylindrical flat-bottomed glass vessels; a set of glass reciprocating cylinders; inert fittings and screens that are designed to fit the tops and bottoms of the reciprocating cylinders; and a motor and drive assembly to reciprocate the cylinders vertically inside the vessels and, if desired, index the reciprocating cylinders horizontally to a different row of vessels. Apparatus 4, the flow-through cell, consists of a reservoir and a pump for the dissolution medium; a flow-through cell; and a water bath that maintains the temperature of the dissolution medium.

In addition, the United States Pharmacopeia USP <724> describes three types of apparatus that can be used to develop an appropriate in vitro drug release method for transdermal systems and other dosage forms. Apparatus 5, the paddle over disk assembly, uses the paddle and disk assembly from Apparatus 2, described above, with the addition of a stainless steel disk assembly designed for holding the transdermal system at the bottom of the vessel. Apparatus 6, the cylinder assembly, uses the assembly from Apparatus 1, but the basket and shaft are replaced with a stainless steel cylinder stirring element and the temperature is maintained at 32±0.5° during the test. Apparatus 7, the reciprocating holder assembly, consists of a set of volumetrically calibrated or tared solution containers made of glass or other suitable inert material; a motor and drive assembly to reciprocate the system vertically and to index the system horizontally to a different row of vessels automatically, if desired; and a set of suitable sample holders. In this apparatus, the solution containers are partially immersed in a suitable water bath that permits maintaining the temperature inside the containers.

Dissolution testing of tablets and other dosage forms such as capsules has been performed using the prior art methods described above. For example, dissolution testing of zolmitriptan tablets has been conducted using Apparatus 2, listed above, which tests the immediate release of zolmitriptan from 500 mL of 0.1N hydrochloric acid.

With the advent of drug-coated microneedle patches, there is a need for a dissolution test suitable to determine the drug release profile from this new dosage form. However, the prior art dissolution methods are unsatisfactory because, inter alia, they are tailored for the more traditional dosage forms such as tablets and capsules. Thus, there is a need in the art for a dissolution method that can accurately measure the drug released from a microneedle patch.

### SUMMARY

According to the present disclosure, the novel dissolution testing method for drug-coated microneedle patches employs certain aspects of USP <724>, USP <1092>, and EOP2 meeting recommendations (dated May 10, 2017). The novel method can be used to test the dissolution of any bioactive agent from a microneedle patch. In one embodiment, referring to FIG. 5, the method comprises adapting an Apparatus 7 device by substituting the novel sample holder and clip assembly for the reciprocating disk sample holder at the end of the shaft of Apparatus 7. As shown in FIGS. 4 and 5, the drug-containing microneedle patch to be tested is affixed to the outer surface of the sample holder with the array of microneedles having drug pointed away from the surface of the sample holder. A membrane, to simulate skin and as further described herein, is placed over the top of the array and in contact with the microneedles. The clip is then coupled to the sample holder to secure the membrane and array against the outer surface of the sample holder.

The method further comprises immersing the loaded and clipped sample holder in a dissolution medium solution. The shaft is then moved or rotated at a predetermined rate or speed for a predetermined time. Aliquots of the dissolution medium are taken at various time points to determine the amount of drug that has released from the microneedles, through the membrane and into the dissolution medium at each time point sampled. Such data is then used to calculate the rate and extent of dissolution of the drug from the microneedle dosage form. In some aspects, the adapted Apparatus 7 employs one or more sequential media tubes, each containing between 50-200 mL of the dissolution medium.

In another embodiment, the method comprises adapting an Apparatus 6 device by substituting the novel sample holder and clip assembly for the reciprocating disk sample holder at the end of the shaft of Apparatus 6. The other steps of the method are similar to those described above. Aliquots of the dissolution medium are taken at various time points to determine the amount of drug that has released from the microneedles, through the membrane and into the dissolution medium at each time point sampled. Such data is then used to calculate the rate and extent of dissolution of the drug from the microneedle dosage form. In certain embodiments, the dissolution medium is placed in a vessel having a volume of about 900 mL. The dissolution medium may be equilibrated to 32 degrees.

In another embodiment of the present disclosure, the method is designed to achieve a desirable multipoint in vitro release profile of a bioactive agent from a microneedle patch with less than about 30% of the drug released at the first time point, and more than about 80% of the drug released at the last time point. In other aspects, the release profile is less than about 20%-40% drug released at the first time point, and more than about 60%-90% drug released at the second time point.

Additional embodiments of the present devices, compositions, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The foregoing features of embodiments will be more readily understood by reference to the following detailed description, taken with reference to the accompanying drawings, in which:

FIG. 1 is a series of line graphs the in vitro released profiles of ZP-Zolmitriptan 1.9 mg patches (TR-5270) using Apparatus 5.

FIG. 2 illustrates Reciprocating Apparatus 7, the reciprocating holder, with 50 mL vessels.

FIG. 3 illustrates a microneedle patch, namely the M207 microneedle patch.

FIG. 4 illustrates the custom sample holder, clip (left) and the custom sample holder containing patch (right), which are used to mount the microneedle patch on Apparatus 7.

FIG. 5 illustrates how the sample is attached to Apparatus 7.

FIG. 6 are photographs of two versions of the custom sample holder and clip.

On the left, the sample holder and clip (revision A) were made from polytetrafluoroethylene (PTFE). On the right of the image, the sample holder and clip (revision B) were made using polyetheretherketone (PEEK).

FIG. 7 is a line graph of the in vitro release profiles of M207 patches in phosphate buffered saline (PBS) from different membranes.

FIG. 8 is a line graph of the in vitro drug release profiles from: (a) spiked unformed microneedle array patches; and (b) patches with formed microneedle array.

FIG. 9 is a line graph of the in vitro release profiles of zolmitriptan released in phosphate buffered saline (PBS) from two drug coated patch lots.

FIG. 10 is a line graph of the in vitro drug release profiles of zolmitriptan in phosphate buffered saline (PBS) at different pH values.

FIG. 11 is a line graph of the in vitro drug release profiles of zolmitriptan in phosphate buffered saline (PBS) at different release media concentration levels.

FIG. 12 is a line graph of the in vitro drug release profiles of zolmitriptan from different dipping rates.

FIG. 13 is a line graph of the in vitro drug release profiles of zolmitriptan at two different bath temperatures (32° C. and 37° C.).

FIG. 14 is a line graph of the different in vitro drug release profiles of zolmitriptan from patches processed with different gap settings.

FIG. 15 is a line graph of the different in vitro drug release profiles of zolmitriptan from patches processed with different arm settings.

FIG. 16 is a line graph of the different in vitro drug release profiles of zolmitriptan from patches processed with different pause settings.

FIG. 17 illustrates the Zolmitriptan UV Spectrum obtained from RP-HPLC.

FIG. 18 depicts the chromatogram of 1×DPBS buffer (reference: 0194-CL-023). DPBS buffer is used as the mobile phase component and does not interfere with the zolmitriptan peak.

FIG. 19 is a line graph of a calibration plot obtained from plotting the response area against the concentration of a series of zolmitriptan USP standard solutions with different concentrations (Linearity Plot).

### DETAILED DESCRIPTION

Various aspects and embodiments will be described herein. These aspects and embodiments may, however, be embodied in many different forms and should not be construed as limiting; rather, these embodiments are provided so the disclosure will be as thorough and complete so as to inform a person of skill how to make and use the compositions, devices, methods of treatment, kits and methods of manufacture of pharmaceutical products described herein. The terminology used herein is for the purpose of describing the compositions, devices, methods of treatment, kits and methods of manufacture described herein, and is not intended to be limiting unless expressly stated, because the scope of the invention will be limited only by claims accompanying this application and claims accompanying continuation and divisional applications derived from this application. All books, publications, patents, and patent applications cited herein are hereby incorporated by reference in their entirety.

As can be appreciated from the foregoing and following description, each and every feature described herein, and each and every combination of two or more of such features, is included within the scope of the present disclosure provided that the features included in such a combination are not mutually inconsistent. For example, any embodiment whose use is consistent with any other embodiment is contemplated and thus included in this description. Other aspects and embodiments are set forth in the following description and claims, and also when considered in conjunction with the accompanying examples and drawings.

As used in this specification and the appended claims, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure.

### A. Definitions

Unless defined otherwise, all terms and phrases used herein include the meanings that the terms and phrases have attained in the art, unless the contrary is clearly indicated or clearly apparent from the context in which the term or phrase is used. Any methods and materials similar or equivalent to those described herein can be used in the practice or testing of the present invention, including the particular methods and materials described herein.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon factors known to those skilled in the art. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden or narrow a particular numerical value or range. As a general matter, “about” or “approximately” broaden the numerical value. The disclosure of ranges is intended as a continuous range including every value between the minimum and maximum values plus the broadening of the range afforded by the use of the term “about” or “approximately.” Consequently, recitation of ranges of values herein are intended to serve as a shorthand method of referring individually to each separate value falling within the range, and each separate value is incorporated into the specification as if it were individually recited herein.

The term “bioactive agent” or “active agent” or “drug” as used herein, means an active pharmaceutical ingredient, drug, therapeutic agent, antigen, drug, peptide, polypeptide or protein. The in vitro dissolution test method described herein is applicable to any drug or formulation that can be coated on a microneedle. Such drugs and formulations are described in, for example, The Merck Index, 15^{th} ed. 2013; Goodman & Gilman's The Pharmacologic Basis of Therapeutics, 13^{th} ed. 2017; American Hospital Formulary Service, Drug Information, 2020 ed.; American Hospital Formulary Service, Handbook of Injectable Drugs, 20^{th} ed. In some embodiments, the bioactive agent present in the microneedle patch dosage form to be tested is a weakly basic drug substance with pKa range of about 8 to 9.9.

The word “intracutaneous” or “transdermal” as used herein, is a generic term that refers to delivery of an active agent (e.g., a therapeutic agent, such as an antigen, a drug, pharmaceutical, peptide, polypeptide or protein) through the skin to the local tissue or systemic circulatory system without substantial cutting or penetration of the skin, such as cutting with a surgical knife or piercing the skin with a hypodermic needle. Intracutaneous agent delivery includes delivery via passive diffusion as well as delivery based upon external energy sources, such as electricity (e.g., iontophoresis) and ultrasound (e.g., phonophoresis).

The term “intracutaneous flux” or “transdermal flux” as used herein, means the rate of intracutaneous or transdermal delivery of an active agent or drug.

The term “microprojection member” or “microneedle array,” and the like as used herein, generally connotes a microprojection grouping comprising a plurality of microprojections, preferably arranged in an array, for penetrating or piercing the stratum corneum. The microprojection member can be formed by etching or punching a plurality of microprojections from a thin sheet of metal or other rigid material, and folding or bending the microprojections out of the plane of the sheet to form a configuration. The microprojection member could alternatively be fabricated with other materials, including plastics or polymers, such as polyetheretherketone (PEEK). The microprojection member can be formed in other known techniques, such as injecting molding or micro-molding, microelectromechanical systems (MEMS), or by forming one or more strips having microprojections along an edge of each of the strip(s), as disclosed in U.S. Pat. Nos. 6,083,196; 6,091,975; 6,050,988; 6,855,131; 8,753,318; 9,387,315; 9,192,749; 7,963,935; 7,556,821; 9,295,714; 8,361,022; 8,633,159; 7,419,481; 7,131,960; 7,798,987; 7,097,631; 9,421,351; 6,953,589; 6,322,808; 6,083,196; 6,855,372; 7,435,299; 7,087,035; 7,184,826; 7,537,795; 8,663,155, and U.S. Pub. Nos. US20080039775; US20150038897; US20160074644; and US20020016562. As will be appreciated by one having ordinary skill in the art, when a microprojection array is employed, the dose of the therapeutic agent that is delivered can also be varied or manipulated by altering the microprojection array size, density, etc.

The term “microprojections” and “microneedles,” as used interchangeably herein, refers to piercing elements that are adapted to penetrate, pierce or cut into and/or through the stratum corneum into the underlying epidermis layer, or epidermis and dermis layers, of the skin of a living animal, particularly a mammal and, more particularly, a human. In one embodiment of the invention, the piercing elements have a projection length less than 1000 microns. In a further embodiment, the piercing elements have a projection length of less than 500 microns, more preferably less than 400 microns. The microprojections further have a width in the range of approximately 25 to 500 microns and a thickness in the range of approximately 10 to 100 microns. The microprojections may be formed in different shapes, such as needles, blades, pins, punches, and combinations thereof.

A bioactive agent “release rate,” as used herein, refers to the quantity of agent released from a dosage form or pharmaceutical composition per unit time, e.g., micrograms of agent released per hour (mcg/hr) or milligrams of agent released per hour (mg/hr). Agent release rates for dosage forms are typically measured as an in vitro rate of dissolution, i.e., a quantity of agent released from the dosage form or pharmaceutical composition per unit time measured under appropriate conditions and in a suitable fluid.

The term “stable,” as used herein, refers to an agent formulation, means the agent formulation is not subject to undue chemical or physical change, including decomposition, breakdown, or inactivation. “Stable” as used herein, refers to a coating also means mechanically stable, i.e., not subject to undue displacement or loss from the surface upon which the coating is deposited.

The term “therapeutically effective” or “therapeutically effective amount,” as used herein, refer to the amount of the biologically active agent needed to stimulate or initiate the desired beneficial result. The amount of the biologically active agent employed in the coatings of the invention will be that amount necessary to deliver an amount of the biologically active agent needed to achieve the desired result. In practice, this will vary widely depending upon the particular biologically active agent being delivered, the site of delivery, and the dissolution and release kinetics for delivery of the biologically active agent into skin tissues.

### B. Novel Dissolution Test for Drug-Coated Microneedle Patch

Drug-coated microneedle patches are used to intracutaneously deliver bioactive agents by piercing the stratum corneum and delivering the drug into the interstitial space. A microneedle patch is typically a single-entity combination drug/device product comprising a disposable patch centered on an adhesive backing with a titanium microneedle array that is coated with a bioactive agent formulation.

According to the present disclosure, the novel dissolution testing method for drug-coated microneedle patches employs certain aspects of USP <724>, USP <1092>, and EOP2 meeting recommendations (dated May 10, 2017). The novel method can be used to test the dissolution of any bioactive agent from a microneedle patch. In some non-limiting embodiments, the method is particularly well-suited to test drugs such as a triptan, e.g., zolmitriptan.

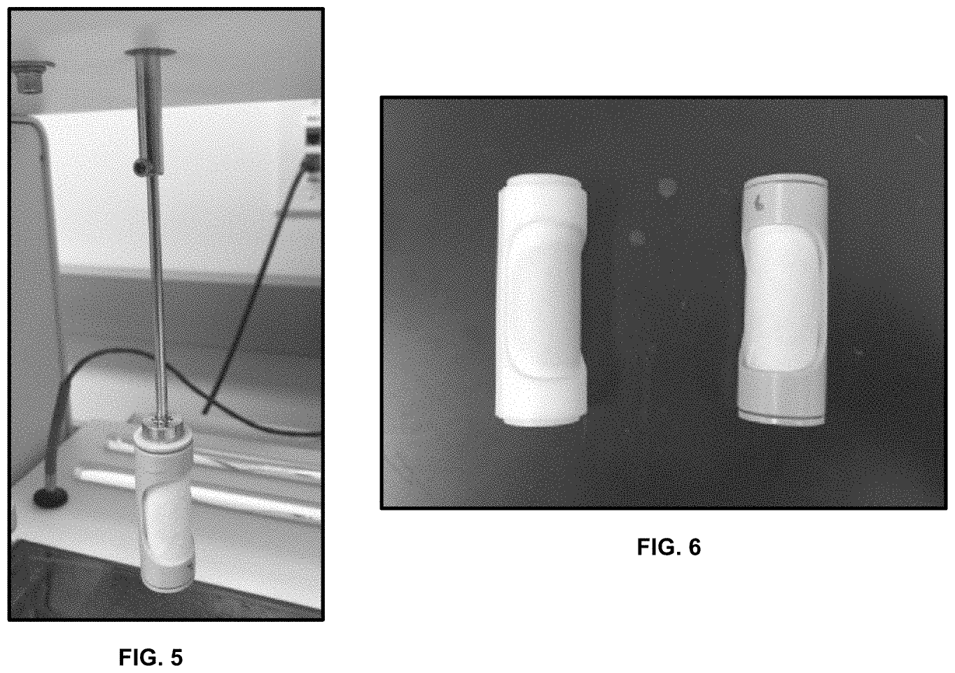

In one embodiment, referring to FIG. 5, the method comprises adapting an Apparatus 7 device by substituting the novel sample holder and clip assembly for the reciprocating disk sample holder at the end of the shaft of Apparatus 7. As shown in FIGS. 4 and 5, the drug-containing microneedle patch to be tested is affixed to the outer surface of the sample holder with the array of microneedles having drug pointed away from the surface of the sample holder. A membrane, to simulate skin and as further described herein, is placed over the top of the array and in contact with the microneedles. The clip is then coupled to the sample holder to secure the membrane and array against the outer surface of the sample holder. Next, the loaded and clipped sample holder is submerged in a dissolution medium solution, and the shaft is moved or rotated at a predetermined rate or speed for a predetermined time. Aliquots of the dissolution medium are taken at various time points to determine the amount of drug that has released from the microneedles, through the membrane and into the dissolution medium at each time point sampled. Such data is then used to calculate the rate and extent of dissolution of the drug from the microneedle dosage form. In some aspects, the adapted Apparatus 7 employs one or more sequential media tubes, each containing between 50-200 mL of the dissolution medium.

In another embodiment, the method comprises adapting an Apparatus 6 device by substituting the novel sample holder and clip assembly for the reciprocating disk sample holder at the end of the shaft of Apparatus 6. The other steps of the method are similar to those described above. Aliquots of the dissolution medium are taken at various time points to determine the amount of drug that has released from the microneedles, through the membrane and into the dissolution medium at each time point sampled. Such data is then used to calculate the rate and extent of dissolution of the drug from the microneedle dosage form. In certain embodiments, the dissolution medium is placed in a vessel having a volume of about 900 mL. The dissolution medium may be equilibrated to 32 degrees.

In an embodiment of the present disclosure, the method is designed to achieve a desirable multipoint in vitro release profile of a bioactive agent from a microneedle patch with less than about 30% of the drug released at the first time point, and more than about 80% of the drug released at the last time point. In other aspects, the release profile is less than about 20%-40% drug released at the first time point, and more than about 60%-90% drug released at the second time point.

Although not strictly required, the first time point is longer than about 15 minutes to ensure that temperature equilibrium is reached between product and release medium. The total testing time for release may be as short as possible to achieve fast turnaround time for quality control (QC) lot release testing, although about 10-40 hours testing time is usually acceptable. In some embodiments, the test method yields data with reasonable variability having a relative standard deviation (RSD) from the first time point being about <20% and a RSD from other time points being about <10% for a sample size of 12.

In one particular embodiment, to determine the in vitro drug release of drug-coated microneedle patches with small amounts of drug coated on the microneedle patches, Apparatus 7 is used with some major modifications. The patches are attached, drug side out, to custom-designed sample holders. The membrane, which mimics the skin, is placed on top of each patch in the holder and firmly held by a clip.

The holders loaded with patches are then mounted to Apparatus 7 and dipped into test tubes, which contain 30 mL of the selected dissolution medium that is maintained at a constant temperature of 32±0.5° C. The samples are next dipped into different tubes at a selected dip rate (dpm) according to the testing interval.

At the end of each dipping interval, the drug release media is transferred to HPLC vials and analyzed for drug content by reverse-phase HPLC. The amount of drug release at each sample collection time interval is added to the total amount of drug released from all the previous time intervals and the % Label Claim of cumulative drug release is reported for each patch. Five calibration standard levels are used to quantify the amount of drug released. System suitability is performed before every HPLC run.

As described in the Example below, a feasibility study can be performed using the method of the present disclosure to determine the rate and extent of release for a bioactive agent. Such study may comprise the steps of: (1) selection of an analytical method for drug content determination; (2) performance of a solubility study; (3) sink condition determination; (4) selection of an apparatus; (5) a sample holder; (6) selection of a membrane; (7) selection of a release medium; (8) selection of the release medium pH and concentration; (9) selection of time points; (10) an agitation study; (11) a bath temperature study; (12) a stability study of the bioactive agent in the release medium; and (13) a discrimination power study. Each step is described below. A person of ordinary skill in the art will appreciate that not all of these steps is necessarily required to determine the rate and extent of release.

1. Analytical Method for Drug Content Determination

High performance liquid chromatography (HPLC) is one of the most common analytical methods used to determine drug content. The drug-specific HPLC parameters should be adjusted to improve detection sensitivity of the drug released. The HPLC parameters that should be adjusted include: the column, the column temperature, the mobile phases, the flow rate, the injection volume, the sample temperature, the detection wavelength, and the run time.

2. Solubility Study

A solubility study of the bioactive agent should be performed at different pH levels. The effect of pH on the bioactive agent should be determined in solutions of: HCL, NaOH, phosphate buffered saline (PBS), water, and isopropyl alcohol.

3. Sink Condition

The sink condition is defined as having a volume of medium at least three times that required to form a saturated solution of drug substance. Sink conditions ensure that the amount of drug dissolved in the medium does not affect the release rate as the drug release progresses. The microneedle patch is coated with a specific amount (mg) of the bioactive agent and is intended to deliver a certain percentage of the drug. Based on the data from the solubility study described above, the sink condition can be met by various media.

4. Apparatus

The United States Pharmacopeia USP <724> describes three types of apparatus that can be used to develop an appropriate in vitro method for transdermal systems and other dosage forms. Current compendial apparatus include the paddle over disk/disk assembly (Apparatus 5), the rotating cylinder with vessel assembly (Apparatus 6), and the reciprocated holder with different rows of vessel assembly (Apparatus 7).

Apparatus 6 typically works for larger patches, but is not suitable for microneedle patches with low amounts of drug coated on the patches. Apparatus 7 is the most appropriate system to test the in vitro drug release profile of microneedle patches with low amounts of bioactive agents coated on the patches. Since Apparatus 7 permits the use of a small release medium volume (<50 mL), this testing method provides greater drug quantitative sensitivity over the duration of testing. In addition, Apparatus 7 works with a wide variety of sample holders such as reciprocating disk, angled disk, cylinder, or spring holder which provides more options for drug-coated microneedle patch testing. Apparatus 7 must be modified substantially to effectively measures the drug release profile from microneedles.

a. Apparatus 5

USP <724> describes Apparatus 5 as follows. The paddle and vessel assembly from Apparatus 2 is used, with the addition of a stainless steel disk assembly designed for holding the transdermal system at the bottom of the vessel. Other appropriate devices may be used, provided that they do not sorb, react with, or interfere with the specimen being tested. The temperature is maintained at 32±0.5°. A distance of 25±2 mm between the paddle blade and the surface of the disk assembly is maintained during the test. The vessel may be covered during the test to minimize evaporation. The disk assembly for holding the transdermal system is designed to minimize any “dead” volume between the disk assembly and the bottom of the vessel. The disk assembly holds the system flat and is positioned such that the release surface is parallel with the bottom of the paddle blade.

To conduct the procedure, dissolution medium is placed in the vessel, the apparatus is assembled without the disk assembly, and the medium is equilibrated to 32±0.5°. The transdermal system is applied to the disk assembly, assuring that the release surface of the system is as flat as possible. The system may be attached to the disk by applying a suitable adhesive to the disk assembly. The system is pressed, release surface side up, onto the adhesive-coated side of the disk assembly.

If a membrane is used to support the system, it is applied so that no air bubbles occur between the membrane and the release surface. The disk assembly is then placed flat at the bottom of the vessel with the release surface facing up and parallel to the edge of the paddle blade and surface of the dissolution medium. The bottom edge of the paddle is 25±2 mm from the surface of the disk assembly.

The apparatus is then immediately operated at the rate specified in the monograph. At each sampling time interval, a specimen is withdrawn from a zone midway between the surface of the dissolution medium and the top of the blade, not less than 1 cm from the vessel wall. Analysis is performed on each sampled aliquot as directed in the individual monograph and volume losses are corrected as necessary. The test is repeated for additional transdermal systems.

The test time points, generally three, are expressed in hours. Specimens are to be withdrawn within a tolerance of ±15 minutes or ±2% of the stated time, the tolerance that results in the narrowest time interval should be selected.

b. Apparatus 6

USP <724> describes Apparatus 6 as follows. The vessel assembly from Apparatus 1 is used, except the basket and shaft are replaced with a stainless steel cylinder stirring element and the temperature is maintained at 32±0.5° during the test. The dosage unit is placed on the cylinder at the beginning of each test. The distance between the inside bottom of the vessel and the cylinder is maintained at 25±2 mm during the test.

The dissolution medium is placed in the vessel of the apparatus specified in the individual monograph, the apparatus is assembled, and the dissolution medium is equilibrated to 32±0.5°. Unless otherwise directed in the individual monograph, the protective liner is removed from the system, and the adhesive side is placed on a piece of Cuprophan that is not less than 1 cm larger on all sides than the system.

The system is then placed Cuprophan covered side down, on a clean surface, and a suitable adhesive is applied to the exposed Cuprophan borders. If necessary, additional adhesives are applied to the back of the system. The adhesive-coated side of the system is then carefully applied to the exterior of the cylinder such that the long axis of system fits around the circumference of the cylinder. The Cuprophan covered is pressed to remove trapped air bubbles. The cylinder is then placed in the apparatus, and immediately rotated at the rate specified in each monograph. Within the specified time interval, a quantity of the dissolution medium is withdrawn for analysis from a zone midway between the surface of the dissolution medium and the top of the rotating cylinder, not less than 1 cm from the vessel wall. Analysis is performed as directed in the individual monograph, and any volume losses are corrected as necessary. The test is repeated for any additional transdermal drug delivery systems.

The test time points, generally three, are expressed in hours. Specimens are to be withdrawn within a tolerance of ±15 minutes or ±2% of the stated time, the tolerance that results in the narrowest time interval being selected.

c. Apparatus 7

USP <724> describes Apparatus 7 as follows. The assembly consists of a set of volumetrically calibrated or tared solution containers made of glass or other suitable inert material; a motor and drive assembly to reciprocate the system vertically and to index the system horizontally to a different row of vessels automatically, if desired; and a set of suitable sample holders. The solution containers are partially immersed in a suitable water bath of any convenient size that permits maintaining the temperature inside the containers at 32±0.5° or within the allowable range, as specified in the individual monograph, during the test. No part of the assembly, including the environment in which the assembly is placed, contributes significant motion, agitation, or vibration beyond that due to the smooth, vertically reciprocating sample holder. Apparatus that permits observation of the system and holder during the test is preferable. The size of the container and the sample holder that should be used can be obtained from the individual monograph.

To prepare a transdermal drug delivery system, the system is pressed onto a dry, unused piece of Cuprophan, nylon netting, or equivalent with the adhesive side against the selected substrate. Care should be taken to eliminate the air bubbles between the substrate and the release surface. The system is attached to a suitable sized sample holder with a suitable O-ring such that the back of the system is adjacent to and centered on the bottom of the disk-shaped sample holder or centered around the circumference of the cylindrical-shaped sample holder. For other drug delivery systems, each system to be tested can be attached to a suitable holder.

To conduct the test, each sample holder is suspended from a vertically reciprocating shaker such that each system is continuously immersed in an accurately measured volume of dissolution medium within a calibrated container pre-equilibrated to temperature, T. Reciprocation then occurs at a frequency of about 30 cycles per minute with an amplitude of about 2 cm, or as specified in the individual monograph, for the specified time in the medium specified for each time point. The solution containers are then removed from the bath, cooled to room temperature, and a sufficient solution is added to correct for evaporative losses. Analysis is performed as directed in the individual monograph. The test is repeated for additional drug delivery systems.

5. Sample Holder and Clip

A sample holder is needed for the samples to be mounted into the apparatus system for analysis. For example, it is mounted as shown in FIG. 5. Since the microneedles are formed at 90 degrees vertically to the surface of the patch, it is difficult to affix the patch to the membrane using the commercially available reciprocating disk sample holder, due to the limited adhesive surface area of microneedle patches.

In order for the drug-coated microneedle patches to be mounted onto Apparatus 7 for drug release testing, the patch with the needles facing out, is attached to an appropriate membrane, as determined below. As shown in FIGS. 4-6, the patch and membrane are wrapped around the sample holder as the outside layer, and a clip is snapped onto the sample holder to secure the patch and the membrane in place. The clip has an opening to allow exposure of the dissolution media to the patch/membrane combination. The sample holder and the patch can be mounted onto the selected apparatus.

In one embodiment, the sample holder is generally cylindrical to facilitate rotation of the sample holder and clip assembly. However, other shapes are contemplated by the present invention such as rectangular, square, oval or any other shape that allows for the rotation of the sample holder clip assembly during the operation of the test method.

While sample holders and clips made using polytetrafluoroethylene (PTFE) become soft and cannot hold patches firmly, sample holders and clips made using polyetheretherketone (PEEK) perform well. As can be seen in FIGS. 4-6, the sample holder and clip assembly provide for an opening of sufficient size to permit testing of different sizes of microneedle patches and allow for the entire surface area of the patch covered by the membrane be immersed in the dissolution media. In some embodiments, the opening of the sample holder may be a variety of shapes, such as rectangular, square, oblong, circular, parallelogram, pentagon, octagon, hexagon, rhombus, oval, or triangular. And, such opening may be of a size of about 10 cm², or 9 cm², or 8 cm², or 7 cm², or 6 cm², or 5 cm², or 4 cm², or 3 cm², or 2 cm², or 1 cm².

6. Membrane

As used herein, the membrane covers the patch affixed to the outer surface of the sample holder. The microneedles are oriented outward from the sample holder so that they come in contact with and pierce (partially or fully) the membrane. Such microneedle patches are designed to deliver a bioactive agent into the interstitial space, upon application to the skin. The drug dissolves off the needles by diffusion; therefore, the need for a membrane is important in attempting to mimic the delivery mechanism. To slow down the drug release and obtain a multipoint release profile, a suitable membrane can provide a degree of hydrodynamic damping.

The membrane can act as an artificial barrier and mimic how the product is actually used. The microneedles contact the membrane, which mimics the skin surface, and subsequently, the drug dissolves from the microneedles and diffuses through the membrane into the release medium.

Membranes suitable for use in the present disclosure include: (1) Whatman Cyclopore track etched membrane, 0.4 um polyester, Cat No: 7061-4704; (2) Millipore Durapore membrane, 0.45 um HV, Cat No: HVLP09050; (3) Millipore Strat-M membrane, REF: SKBM04760; (4) Agilent Cuprophan membrane, Part No: 12-1370; and (5) Whatman Phase separator (PS) paper, cat No: 2200-090.

7. Release Medium

The release medium should be selected next. Phosphate buffered saline (PBS) is the typical media used to determine drug release from transdermal systems. The sink condition of the bioactive agent must be attained in PBS before PBS is selected as the release medium for the drug product.

8. Release Medium PH and Concentration

The effect of the selected release medium pH on the drug product release profile should then be evaluated. Using pH values of 7.2, 7.4, and 7.9, percent drug release should be calculated based on the label claim, which lists how much bioactive agent (mg) is coated on the microneedle.

The effect of the release medium concentration on the drug release profile in the selected medium should also be studied. Percentage drug release can be calculated based on the label claim of the bioactive agent. Medium concentrations of 0.5×, 1×, and 1.5× should be used in the analysis.

9. Time Point Selection

To provide a thorough characterization of the bioactive agent's release, six time points should be collected during method development. The first time point should occur where less than 30% of the drug is released, while more than 80% of the drug should be released at the last time point.

10. Agitation Study

Next, the effect of the dip rate on the drug release profile should be evaluated during method development in order to obtain an optimal dip rate. Approximately six samples from one lot of the drug-coated microneedle patch should be tested at 5, 30, and 60 dpm in 30 mL of the selected release medium at a temperature of 32° C. Percent drug release can be calculated based on the bioactive agent's label claim.

11. Bath Temperature

For the in vitro drug release test for transdermal systems, the solution containers are immersed in a water bath that maintains the temperature inside the containers at 32±0.5° C. per USP <724>. In vitro drug release profiles of the bioactive agent from the patches should be obtained at a bath temperature of 37° C. for comparison purposes during method development.

12. Stability of Bioactive Agent in Release Medium

The bioactive agent's stability in the release medium should be evaluated for up to the last selected time point (hours). This last time point should be where more than 80% of the drug has been released into the medium.

13. Discrimination Power Study

To evaluate the discriminatory power of the method, drug product with different process parameter setting should be manufactured and tested. The percentage of drug release for each time point should be calculated against the drug label claim.

The process for coating microneedle patches involve dipping microneedles into a drug reservoir for a given duration to obtain the target content amount. The patches are coated by passing the microneedles across a rotating drum with a uniform film coating. The amount of drug deposited onto the microneedles can be tuned by adjusting coating parameters. The content amount is controlled by tuning the dipping process parameters for the number of passes, coating gap, pause time, arm speed, and drum speed. Maintaining a uniform and consistent coating is dependent on tight control over parameters.

### EXAMPLES

### Example 1—Analytical Method for Determination of In Vitro Release Profile of Zolmitriptan for M207 Patches

The Zosano Intracutaneous Microneedle System M207 is a single-entity combination drug/device product which consists of a disposable patch centered on an adhesive backing with a titanium microneedle array that is coated with the zolmitriptan formulation (45% w/w). A zolmitriptan coating formulation comprises 45% w/w zolmitriptan, 15% w/w tartaric acid and 40% w/w water. The drug coated patch was packaged in a foil pouch (MS-7050, MS-7052) or a foil cup (MS-7053) as primary package for M207 System.

USP monograph of zolmitriptan tablets provides dissolution testing of zolmitriptan per USP <711>, using Apparatus 2, for immediate release of zolmitriptan from 500 mL of 0.1N hydrochloric acid within 15 minutes. FDA dissolution methods database also provides one dissolution method of zolmitriptan similar to the method in USP monograph of zolmitriptan tablets. The dissolution method in the FDA database also applies to zolmitriptan tablet dosage form and uses the same Apparatus and release medium with multiple sample points up to 30 minutes. Both dissolution methods are not suitable for zolmitriptan patches as zolmitriptan instantaneously release from zolmitriptan patches within a minute. In addition, Apparatus 2 is not the appropriate Apparatus system for transdermal system drug released determination.

### Materials

The materials that were used to test the in vitro release profile of zolmitriptan from M207 patches included the following items:
1. 2 mL Polypropylene HPLC vials, Thermo Scientific P/N: C4011-14
2. 11 mm snap cap, clear snap PTFE/red rubber, VWR, Cat No: 46610-718
3. 50 ml centrifuge tube, VWR, 93000-032
4. Class A glass pipette, 1 mL, 2 mL, 3 mL, 4 mL, 25 mL
5 Dulbecco's Phosphate-Buffered Saline (DPBS), 10×, Alfa Aesar, lot #Q06D510; lot #Z260504
6. Dulbecco's Phosphate-buffered Saline (DPBS), Corning cellgro, 1×, lot #21031479; VWR, lot #16A110003
7. Glass test tube, 55 ml, 25×150 mm, VWR, cat #89000-490
8. Whatman phase separator, GE Healthcare Life Science, cat #2200-070, lot #9848192; lot #9761234
9. Custom designed sample holders form, Rev. A, Rev. B, part number 1210-003, DLS projects LLC
10. Custom designed sample holder clip, Rev. A, Rev. B, part number 1210-0004, DLS projects LLC
11. Methanol, EMD, P/N:MX0475P-1, lot #57236, expiration date: 12 Jan. 20; lot #55239, expiration date: 18 Feb. 18; lot #56126, expiration date: 3 Oct. 18; lot #57012, expiration: 17 Jul. 19
12. Acetonitrile, J.T. Baker, P/N:9012-03, lot #0000172261, expiration date: 30 Apr. 22; lot #0000128450, expiration date: 18 Feb. 18; lot #0000156758, expiration date: 14 Oct. 21
13. Zolmitriptan Reference Standard: USP Lot R02720, current lot.
14. Ammonium Dihydrogen Phosphate, J.T. Baker, lot #0000104852, expiration date: 8 Oct. 20
15. Ammonium hydroxide, Alfa Aesar, lot #Q13B005, expiration date: 20 Jun. 18; BDH, lot #15L170003, expiration: December 2017
16. HPLC water, J.T. Baker, P/N: 4218-03, lot #0000183754, expiration date: 21 Sep. 21; lot #0000190020, expiration date: 14 Dec. 21; lot #0000184107, expiration date: 26 Sep. 21; EMD, lot #56087, expiration date: 30 Apr. 17; lot #56160, expiration date: 30 Jun. 17; lot #57095, expiration date: 30 Apr. 18; lot #57156, expiration date: 30 Jun. 18
17. Zolmitriptan Patch in pouch, lot #0225P001; lot #0164116; lot #12 Sep. 17; lot #NB0178-120; lot #NB0178-100; lot #17010(PN7050); lot #NB0253-10, lot #NB0253-14
18. Zolmitriptan patch in cup, lot #8 Mar. 18

The equipment used to test the in vitro release profile of zolmitriptan from M207 patches included the following items:
1. Agilent technologies reciprocating holder Apparatus 7, EIN: 21218, calibration due date (Cal due): May2018; May 2019
2. Glass test tube, 55 ml, 25×150 mm, Agilent technologies
3. Custom designed sample holders form, part number 1210-003, DLS projects LLC
4. Custom designed sample holder clip, part number 1210-0004 DLS projects LLC
5. Analytical balance, EIN 21215, Cal due: 31 Sep. 2017; 31 Sep. 2018;
6. Dispenser pipette, EIN:20137, Cal due: August 2018;
7. HPLC system, Waters, EIN: 20039, Cal due: 31 Dec. 16; 31 Dec. 17; EIN: 21219, Cal due: August 2018; EIN: 21221, Cal due: August 2018; EIN: 21223, Cal due: February 2018; February 2019; EIN: 21224, Cal due: February 2018; February 2019
8. HPLC Column, Kinetex, 5 μm, EVO C18, 100 A, 4.6×50 mm, ID: Col-16-5; Col-16-9; Col-16-10
9. Vortex mixer, Baxter, EIN:10258;
10. pH meter Thermo Scientific Orion star A211, EIN: 21216, Cal Due: 30 Sep. 2017; 30 Sep. 2018;
11. Refrigerator maintaining at 2-8° C., EIN:20107, Cal Due: May2017; November 2017, May2018, November 2018

### Feasibility Study Experiments of the Novel Dissolution Method

### 1. Analytical Method for Drug Content Determination

During early development experiments, test method TM-600 “Zolmitriptan Content” was utilized to evaluate the solubility of zolmitriptan in PBS, isopropyl alcohol (IPA) and de-ionized water.

The late stage experiments used the same HPLC method with slight parameters modification. The UV detection wavelength was changed from 280 nm to 225 nm, to improve the detection sensitivity for the drug release method. The zolmitriptan standard calibration curve range was modified to 0.5-40 μg/mL (corresponds to 0.8%-63% of 1.9 mg zolmitriptan) to bracket the lower and upper concentration of drug released in the individual dissolution tubes (Apparatus 7). The typical concentration of drug released from each release time point is within the range of 6-15 μg/mL. The HPLC method parameters are listed in Table 1 below.

HPLC Parameters

| Parameter | Description |
|---|---|
| Column | Kinetex 5 μ EVO C18 100 A, 4.6 x 50 mm |
| Column Temperature | 30° C. ± 5° C. |
| Mobile Phase A | Ammonium Dihydrogen Phosphate buffer: |
| | MeOH: Acetonitrile, 70:20:10 (v/v) |
| Mobile Phase B | Ammonium Dihydrogen Phosphate buffer: |
| | Acetonitrile, 30:70 (v/v) |
| Mobile Phase | Isocratic: Mobile Phase A: Mobile Phase B |
| | (80%:20%) |
| Flow Rate | 1.0 mL/min |
| Injection Volume | 15 μL |
| Sampler Temperature | 5° C. |
| Detection Wavelength | 225 nm (280 nm) |
| Run Time | 2 minutes |

### 2. Solubility Study

The drug substance, Zolmitriptan, USP used in M207 is currently provided by a qualified supplier, INKE, S.P.A. According to the Inke DMF, zolmitriptan is slightly soluble to very slightly soluble in aqueous solution at pH range 4-14, with approximately each gram of solute dissolved in 10,000 mL of solution. The effect of pH on zolmitriptan solubility has been determined in HCl and NaOH solutions by Inke. The results are shown in Table 2.

The solubility of zolmitriptan at room temperature in phosphate buffer saline (PBS), water, and isopropyl alcohol (IPA) were evaluated in-house. Zolmitriptan API was added into 200 mL of water, 10 mM phosphate buffer saline (PBS) at pH 7.4, and IPA until saturated. The solutions were then mixed at room temperature by stir bar overnight. Saturated solutions at 2 and 24 hours were filtered and analyzed for drug concentration by HPLC.

As shown in Table 3, solubility of zolmitriptan in water is approximately 1000 μg/mL and is approximately 1200 μg/mL in 10 mM PBS. Solubility results are consistent with Inke's data. Solubility of zolmitriptan in IPA is approximately 1700 μg/mL, slightly higher than in an aqueous solution.

TABLE 2
Solubility of zolmitriptan at different pHs (Data from Inke, S.A. DMF)

| | | Approximate volume of solvent in |
|---|---|---|
| pH | Descriptive Term | mL per gram of solute |
| 1 | Soluble | From 10 to 30 |
| 2 | Slightly soluble | From 100 to 500 |
| 3 | Practically insoluble | More than 10,000 |
| 4 | Practically insoluble | More than 10,000 |
| 7 | Practically insoluble | More than 10,000 |
| 14 | Practically insoluble | More than 10,000 |

TABLE 3
Solubility Study Summa

| | | Zolmitriptan |
|---|---|---|
| Medium | Mixed Time | Concentration (μg/mL) |
| HPLC grade Water | 2 hours | 944.05 |
| | 24 hours | 1061.33 |
| 10 mM PBS, pH 7.4 | 2 hours | 1236.05 |
| | 24 hours | 1213.19 |
| 100% IPA | 2 hours | 1691.82 |

### 3. Sink Condition

The M207 patch is coated with 1.9 mg of zolmitriptan and is intended to deliver 100% of the drug. The mean drug content specification is 90.0%-110.0% of 1.9 mg. Based on the data from the solubility study, the sink condition was met for both the water and 10 mM PBS medium when applying volume of release medium of >6 mL. This is greater than three times the saturated solution in PBS (˜1.6 mL) for 1.9 mg of zolmitriptan released from the M207 patch.

### 4. Apparatus

a. Apparatus 5

The drug product's in vitro drug release profile was first evaluated using the USP paddle over disk, Apparatus 5. The release profile was generated from a Distek 2100C 6-position release tester.

The paddle height was set at 25 mm above the patch with a rotation speed of 50 RPM. The release medium was 500 mL PBS controlled at a temperature of 32° C. A full patch assembly, (which consists of a coated patch adhered to the center of an inner ring and attached to an outer ring), was inserted to the bottom of vessel with the zolmitriptan-coated microneedles facing upright.

The release of zolmitriptan from the coated patches was continually monitored via quantitation of zolmitriptan concentration of the dissolution medium by UV absorbance using a Pion Rainbow 6-Ch Fiber Optic System with a 14 cm dip probe and 10 mm pathlength. The in vitro drug release profiles from three zolmitriptan lots (Lot #0164004 stored at room temperature for 10 months, Lot #0203149-NI and Lot #0203149-IR stored at 40° C./75% RH for 10 months) were evaluated.

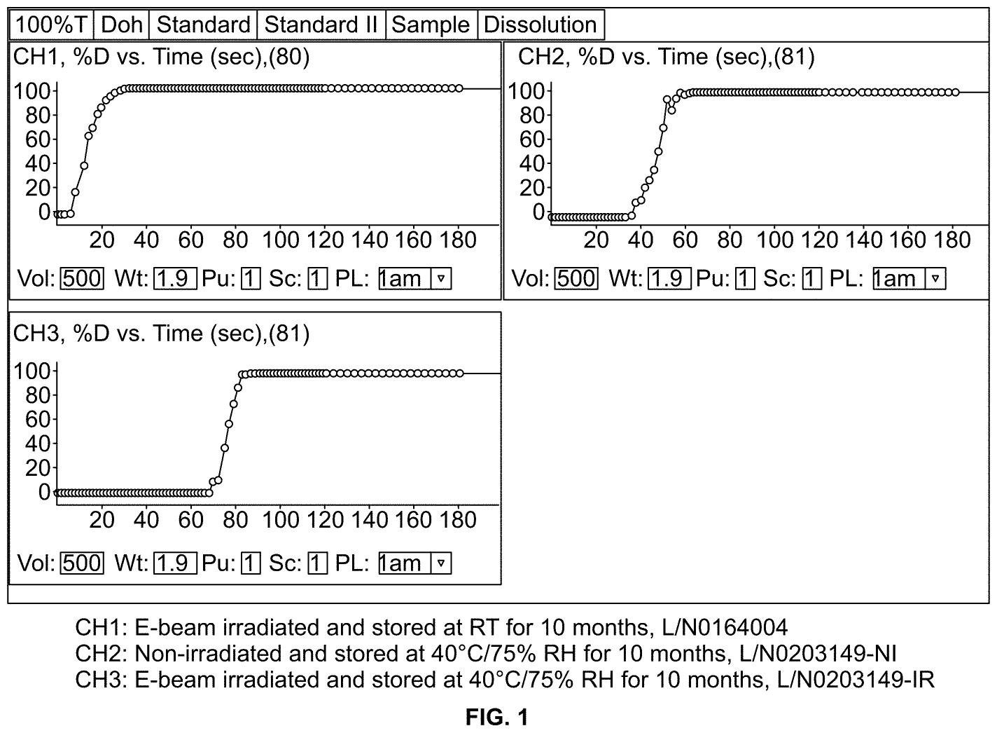

The drug release profiles from Apparatus 5 shown in FIG. 1 indicate an instantaneous release of zolmitriptan for all the patches tested with complete drug release in less than one minute. The high variability among these tested samples was possibly due to the extremely quick release of zolmitriptan coating and renders little discriminatory capability for the drug product. Detailed information of this study is documented in TR-5270.

b. Apparatus 6

Apparatus 6 uses the same vessel assembly as Apparatus 1, except a rotating cylinder is used instead of a basket and shaft. The typical vessel volume is from 1 L to 4 L. Apparatus 6 typically works for larger patches and is not suitable for the M207 patches due to the low amount of coated drug on the patches.

b. Apparatus 7

Apparatus 7 (Reciprocating Holder) is considered an appropriate system for the in vitro drug release profile of M207 patches, as it permits the use of small release medium volume (<50 mL), and therefore, provides greater drug quantitative sensitivity over the duration of testing. Apparatus 7 also works with a wide variety of sample holders such as reciprocating disk, angled disk, cylinder, or spring holder which provides more options for M207 testing.

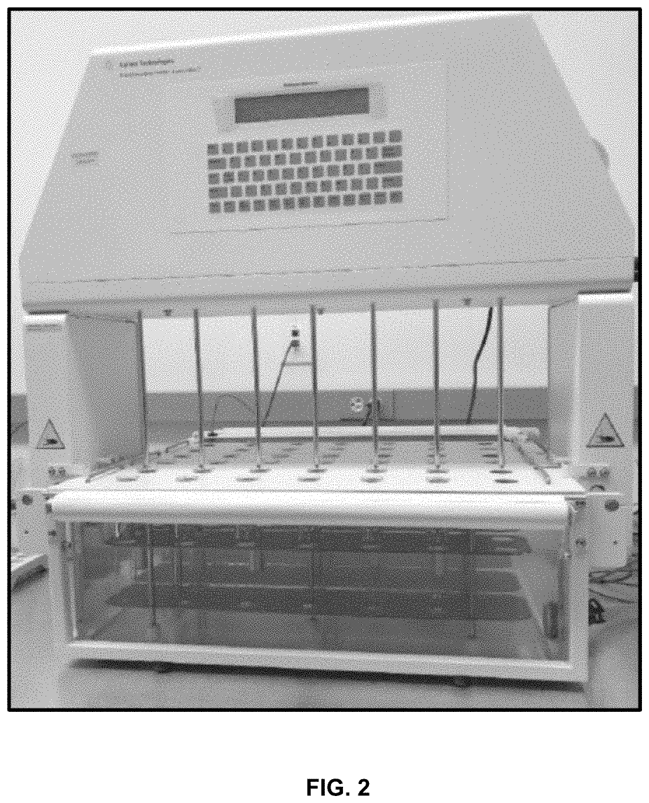

The Agilent Apparatus 7 with the smallest vessel volume of 50 mL, (FIG. 2) was selected for drug release profile testing. Thirty to 50 mL of dissolution media were added to 50 mL vessels for each release time point, not compromising the drug analytical sensitivity. The patches were mounted to the reciprocated holders and dipped in the dissolution media maintained at 32° C. At each time point the samples were advanced to the next row of tubes containing fresh dissolution media. Agitation is described by dips per minutes (DPM).

### 5. Sample Holder

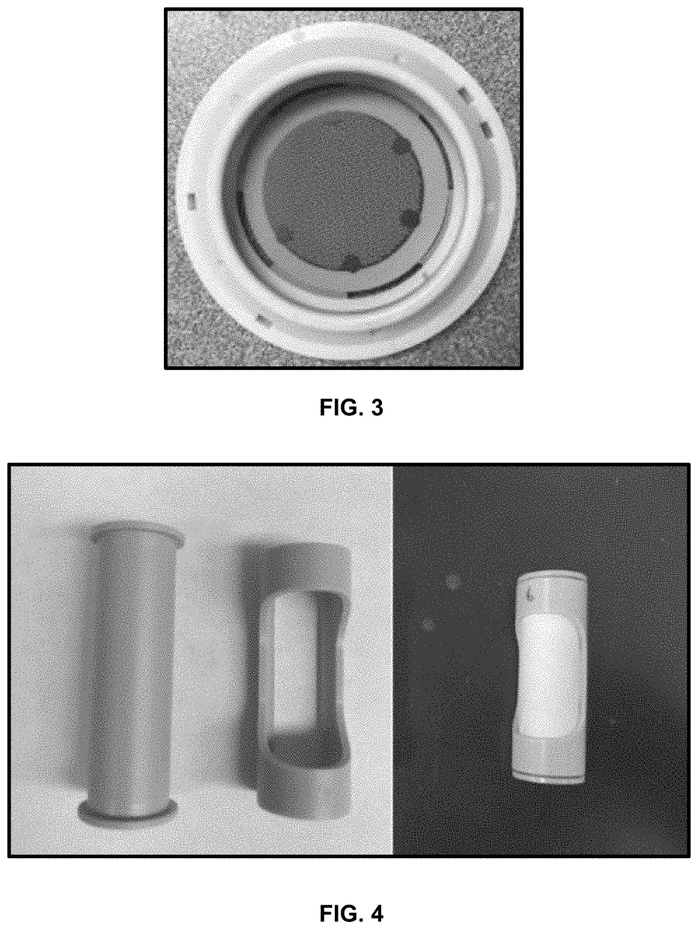

Dimensionally, the M207 patches resemble a 3 cm² titanium array of microneedles coated with drug which are attached to the center of a 5 cm² adhesive patch (FIG. 3).

The 340 micron microneedles are formed at 90 degrees vertically to the surface of patch. It is difficult to affix the patch to the membrane using the commercial reciprocating disk sample holder due to the limited adhesive surface area on M207 patches. In addition, it was difficult to attain a smooth release surface from the patch. After consultation with Agilent technical engineers, a custom sample holder (FIG. 4) was employed to hold the patch to provide a smooth release surface.

In order for the M207 patch to be mounted onto Apparatus 7 for drug release testing, the M207 patch, with needles facing out, was attached to an appropriate membrane (as described in section 6.8). Both patch and membrane were then wrapped around the sample holder with the membrane as the outside layer. A clip was snapped on to the sample holder to secure the patch and membrane in place (FIG. 4). The sample holder containing the patch was mounted onto Apparatus 7 using the reciprocated holder for drug release testing (FIG. 5).

The first version of the sample holder and clip were made from polytetrafluoroethylene (PTFE). It was observed that the clip became soft and could not hold the patches firmly after a few experimental runs. The sample holder and clip were re-designed (Revision B) using Polyetheretherketone (PEEK) as shown in FIG. 6. Revision B of the sample holder and clip are used in the final test method. Refer to FIG. 6 for the comparison of Revision A and Revision B custom sample holders and clips.

### 6. Membrane

In order to slow down the drug release and obtain a multipoint release profile, a suitable membrane was required to provide a degree of hydrodynamic damping. The rationale of incorporating a membrane was to act as an artificial barrier/surrogate to mimic how the product is actually used. The microneedles contact the membrane which mimic the skin surface. The drug will dissolve from microneedles and diffuse through membrane into the release medium. The membranes evaluated were selected based on their properties e.g. hydrophilicity/hydrophobicity, pore size, thickness and their effect on drug release profile assessed.

The following membranes were screened: 1) Whatman Cyclopore track etched membrane, 0.4 um polyester, Cat No: 7061-4704; 2) Millipore Durapore membrane, 0.45 um HV, Cat No: HVLP09050; 3) Millipore Strat-M membrane, REF: SKBM04760; 4) Agilent Cuprophan membrane, Part No: 12-1370; 5) Whatman Phase separator (PS) paper, cat No: 2200-090.

Membrane screening was performed as described below. The M207 patches from lot 0164116 and lot 12 Sep. 17 were attached to the membrane with the array facing the membrane, then wrapped around the sample holder by the clip as shown in FIG. 4. The clip position was adjusted so that the array was not blocked by the clip. The sample holder was mounted to the holder of Apparatus 7. Test tubes containing 30 mL of 10 mM PBS dissolution media at 32° C. were loaded onto Apparatus 7 before the initiation of the experiment (Table 4). At the start of the experiment, samples were dipped at the rate of 30 DPM in the medium for each time interval. At each time point, the concentration of zolmitriptan released into the medium was analyzed by HPLC using the parameters listed in Table 1.

TABLE 4
Drug Released Program

| Apparatus | Apparatus 7 with 50 mL Tubes |
|---|---|
| Sample Holder | Custom design PTFE holder and Clip |
| Medium | 10 mM PBS, pH 7.4 |
| Medium Volume | 30 mL |
| Temperature | 32 ± 0.5° C. |
| DPM | 30 |

With the custom design sample holder and clip, the zolmitriptan-coated microneedles were in contact with the membrane. Upon initiation of the test, the sample holders containing the membrane and coated-microneedles are dipped into the tubes containing the PBS media, so that the outer side of the membrane is wetted by the media. The media then diffused through the membrane, wetting the inner side of the membrane i.e. the microneedle-contacting side. Dissolution of the zolmitriptan from microneedles took place when the inner side of the membrane became imbibed with the media. Thus, the drug release profile was dependent on the release/availability of drug from the microneedles.

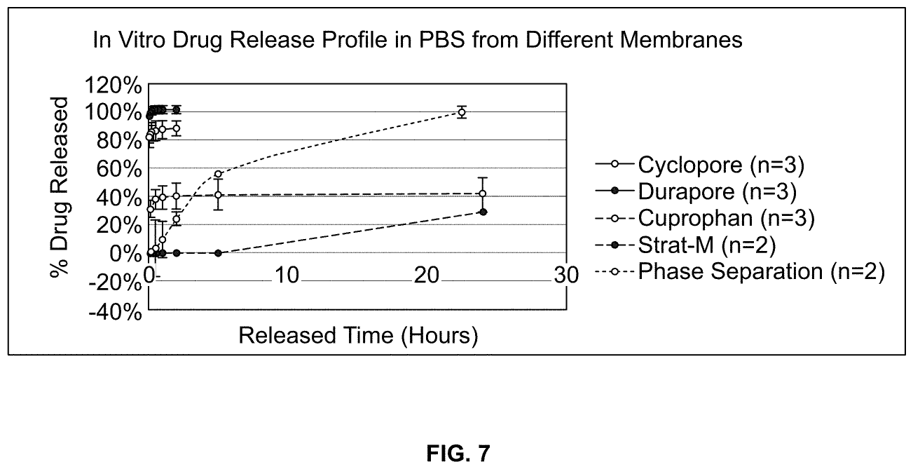

The in vitro drug release profiles of M207 patches from different membranes are summarized in Table 5 and plotted in FIG. 7. Percent drug release was calculated based on the target content of 1.9 mg zolmitriptan coated on each patch. Since the cyclopore membrane consists of a hydrophilic polyester membrane and the durapore membrane is made from a hydrophilic PVDF membrane, the instantaneous release of zolmitriptan through these two membranes was anticipated and observed. Eighty two percent, and 97% of zolmitriptan released within 5 minutes from the cyclopore membrane and durapore membranes and both reached a plateau of drug release within 10 minutes. Approximately 31% of zolmitriptan was released from the cuprophan membrane after 10 minutes and reached a plateau of drug release at approximately 40% after 1 hour.

The release profile of zolmitriptan from the Strat-M membrane behaved opposite to the hydrophilic membrane. The thick polyethersulfone (PES) layers on top of the strat-M membrane inhibited drug release from patches up to 5 hours and approximately 29% was released after 24 hours.

Among the tested membranes, the phase separator appeared to be the most suitable membrane for zolmitriptan in vitro drug release determination. Whatman phase separator (PS) paper is impregnated with stabilized silicone, creating a hydrophobic filter to retain aqueous phases. The thickness of PS membrane is approximately 0.20 mm, which allows zolmitriptan-coated microneedles to contact the membrane, but not punch through it. Approximately 3% of the drug was released at 30 minutes, 9% released at 1 hour and complete drug released at 23 hours. Middle time points, with 40-60% of zolmitriptan released, could be easily optimized between the 4-20 hours window. The phase separator (PS) membrane was selected as the membrane for the M207 patches.

TABLE 5
In vitro Drug Release Summary from Different Membranes
% Drug released/Label Claim (1.17 mg zolmitriptan for patches tested by PS
membrane; 1.9 mg zolmitriptan for patches tested by other membranes)

| | Time | 5 | 10 | 15 | 30 | | |
|---|---|---|---|---|---|---|---|
| Membrane | Point | minutes | minutes | minutes | minutes | 1 hours | 2 hours |
| Cyclopore (n = 3) | Average | 82% | 85% | 86% | 87% | 87% | 88% |
| | RSD | 8% | 7% | 7% | 7% | 6% | 6% |
| Durapore (n = 3) | Average | 97% | 100% | 101% | 101% | 101% | 101% |
| | RSD | 3% | 2% | 2% | 3% | 3% | 3% |
| | Time | 10 | 30 | | | | |
| Membrane | Point | minutes | minutes | 1 hour | 2 hours | 5 hours | 24 hours |
| Cuprophan | Average | 31% | 38% | 39% | 40% | 41% | 42% |
| (n = 3) | RSD | 5% | 7% | 8% | 9% | 10% | 11% |
| Strat-M (n = 2) | Average | 0% | 0% | 0% | 0% | 0% | 29% |
| | Time | 15 | 30 | | | | 22.5 |
| Membrane | Point | minutes | minutes | 1 hour | 2 hours | 5 hours | hours |
| Phase Separator | Average | 1% | 3% | 9% | 24% | 56% | 100% |
| (n = 2) | | | | | | | |

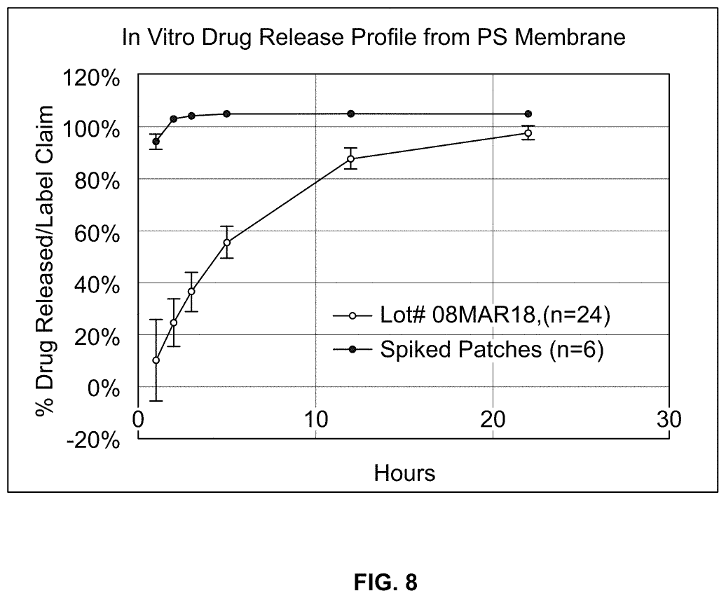

In order to understand how the drug is diffused through the membrane, the in vitro drug release profile of unformed microneedle array patches spiked with 2 mg of zolmitriptan was studied. The summary of in vitro drug release data from spiked patches is provided in Table 6 and plotted in FIG. 8. In vitro drug release data from a lot made from nominal manufacturing process is also listed for comparison. As shown in FIG. 8, more than 90% drug released from the first 1 hour time point and the released profile reached plateau at the 2 hour time point for patches with unformed microneedles. Approximately 10% of the drug was released at the 1 hour time point and 98% of the drug was released at the 22 hour time point for patches with formed microneedles. The multiple time points control release profile is dependent on the diffusion of drug released from formed microneedle. The membrane acted as a surface for microneedles to contact, and then the drug diffused into the media.

TABLE 6
In Vitro Drug Release Summary from Patches with unformed Microneedles
and formed Microneedles from 10 mM PBS Using Phase Separator membrane

| | | | | | | | | Total |
|---|---|---|---|---|---|---|---|---|
| | | | | | | | | Drug |
| | Time | % Drug released/Label Claim | | | | | | Loaded |
| Sample ID | Point | 1 hour | 2 hours | 3 hours | 5 hours | 12 hours | 22 hours | (mg) |
| Unformed | Average | 94% | 103% | 104% | 105% | 105% | 105% | 1.99 |
| microneedle | RSD | 3% | 1% | 0% | 1% | 1% | 1% | 1% |
| array patches | | | | | | | | |
| spiked with | | | | | | | | |
| 2 mg drug | | | | | | | | |
| Lot | Average | 10% | 24% | 37% | 55% | 88% | 98% | 1.95 |
| Mar. 8, 2018 | RSD | 16% | 9% | 8% | 6% | 4% | 3% | |

### 7. Release Medium

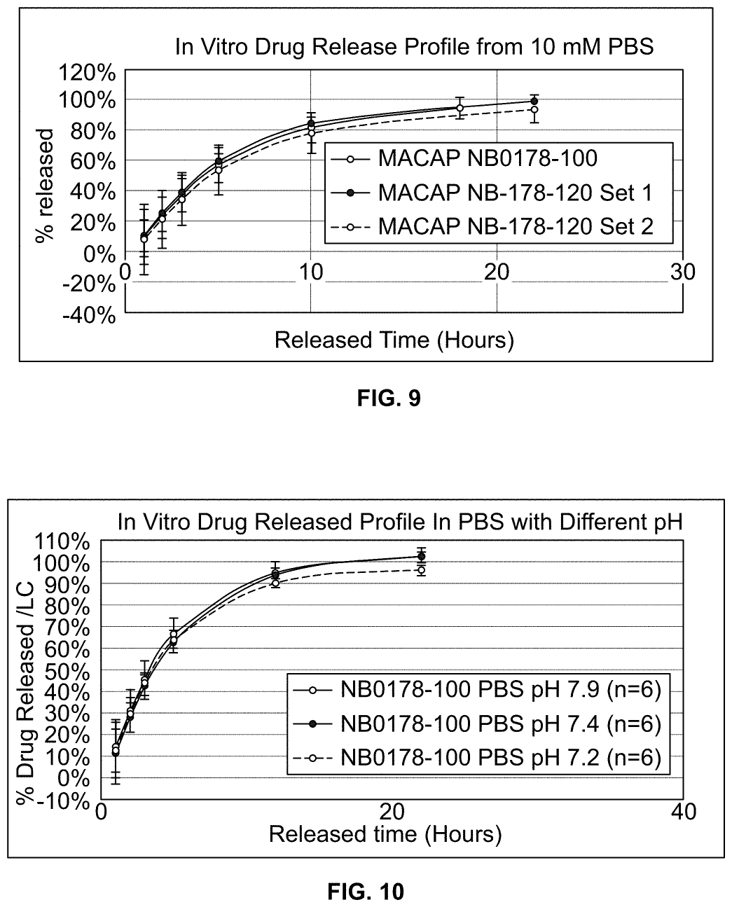

Since the sink condition of zolmitriptan was attained in PBS, PBS is the selected release medium for the drug product. The release profiles of zolmitriptan released in PBS from two drug coated patch lots demonstrated that multipoint release profiles were achieved (Table 7 and FIG. 9). The drug release was less than 30% at the 1 hour time point, and more than 90% after the 18 hour time point for both lots.

TABLE 7
In Vitro Drug Release Summary from 10 mM PBS Using Phase Separator membrane

| | Time | % Drug released/Total Drug Loaded | | | | | |
|---|---|---|---|---|---|---|---|
| Lot # | Point | 1 hour | 2 hours | 3 hours | 5 hours | 10 hours | 18 hours |
| NB0178-100 | Average | 9% | 24% | 38% | 57% | 82% | 95% |
| (n = 6) | RSD | 12% | 11% | 11% | 11% | 9% | 7% |
| | Time | | | | | | |
| | Point | 1 hour | 2 hours | 3 hours | 5 hours | 10 hours | 22 hours |
| NB0178-120 | Average | 10% | 25% | 38% | 59% | 84% | 99% |
| (n = 6)-Analyst 1 | RSD | 18% | 16% | 11% | 5% | 5% | 0% |
| NB0178-120 | Average | 8% | 22% | 34% | 53% | 78% | 94% |
| (n = 6)-Analyst 2 | RSD | 23% | 19% | 18% | 16% | 13% | 9% |

### 8. Release Medium pH and Concentration

The effect of the release media pH on the drug product release profile was evaluated. The drug release program and HPLC parameters used in this study were the same as listed in Table 4 and Table 1 except the pH of the medium was modified. Table 8 and FIG. 10 include the results from 10 mM PBS with different pH conditions. Patches from the same lot, NB0178-100, were used. Percent drug release was calculated based on the label claim of 1.9 mg zolmitriptan. All the results for lot NB0178-100 from 10 mM PBS medium with pH range from 7.2 to 7.9 had similar release profiles. The difference in the mean values was no more than 5%. In addition, the percentage of drug release at each time point were similar, and the drug release was more than 85% at the 12 hour time point. Therefore, the PBS medium with pH of 7.4 was selected as the release medium since no pH adjustment was required during preparation.

TABLE 8
In Vitro Drug Released Summary from PBS with Different pH

| | NB0178- | | NB0178- | | NB0178- | |
|---|---|---|---|---|---|---|
| | 100 PBS pH | | 100 PBS pH | | 100 PBS pH | |
| Time point | 7.9 (n = 6) | RSD | 7.4 (n = 6) | RSD | 7.2 (n = 6) | RSD |
| 1 | 14% | 11% | 12% | 11% | 12% | 15% |
| 2 | 31% | 10% | 28% | 7% | 29% | 8% |
| 3 | 46% | 9% | 43% | 6% | 44% | 5% |
| 5 | 66% | 8% | 63% | 5% | 64% | 3% |
| 12 | 95% | 6% | 94% | 4% | 90% | 1% |
| 22 | 103% | 4% | 103% | 3% | 96% | 2% |

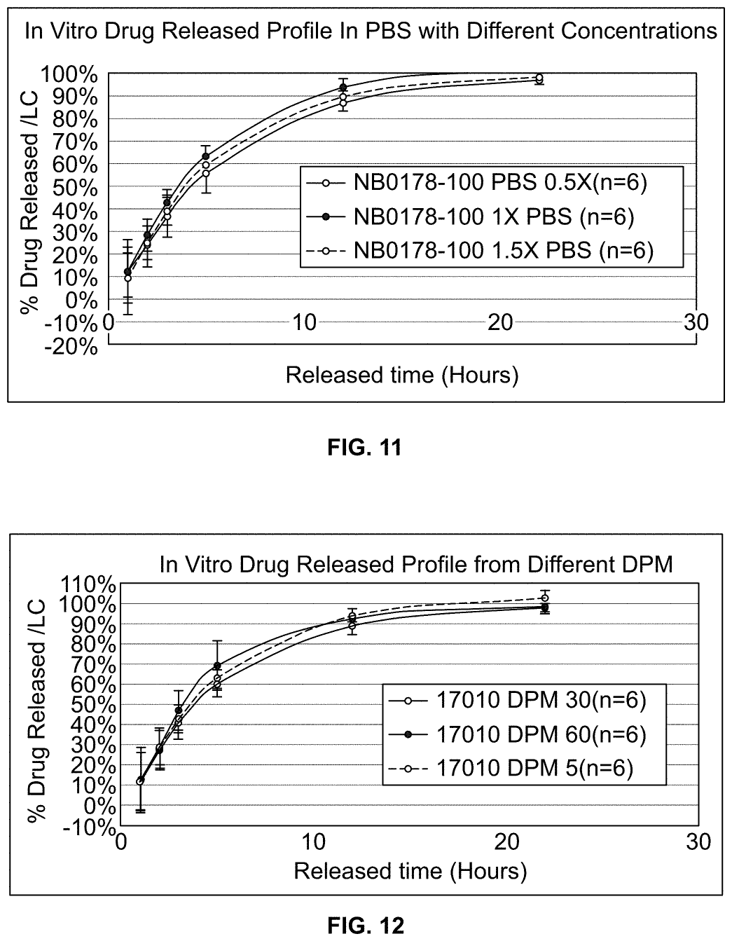

The effect of the release media concentration on the drug release profile in PBS was also studied. Table 9 lists the results and FIG. 11 includes plotted graphs of the release profiles. Percent drug release was calculated based on the 1.9 mg zolmitriptan label claim. Concentration of 1×PBS medium is 10 mM with pH 7.4. This was prepared from a 1 to 10 dilution of stock PBS. The percent drug release within the first two hours with the media concentrations of 0.5×, 1x and 1.5× were similar; percent drug release at the 3 hour and 5 hour time points from 1×PBS medium were slightly faster than the 0.5× and 1.5× media. More than 85% of the drug was released at the 12 hour time point from all three media. Similar amounts were observed from the first two time points for all three concentration media. The 1×PBS medium was selected because more drug was released at the last two time points (12 and 22 hours, respectively).

TABLE 9
In Vitro Drug Released Summary from PBS with Different Concentration

| | NB0178-100 | | NB0178-100 | | NB0178-100 | |
|---|---|---|---|---|---|---|
| | 0.5X PBS | | 1X PBS | | 1.5X PBS | |
| Time point | (n = 6) | RSD | (n = 6) | RSD | (n = 6) | RSD |
| 1 | 10% | 17% | 12% | 11% | 9% | 11% |
| 2 | 24% | 10% | 28% | 7% | 25% | 7% |
| 3 | 37% | 9% | 43% | 6% | 39% | 6% |
| 5 | 56% | 8% | 63% | 5% | 59% | 4% |
| 12 | 87% | 4% | 94% | 4% | 90% | 3% |
| 22 | 98% | 1% | 103% | 3% | 98% | 3% |

### 9. Time Point Selection

To provide a thorough characterization of zolmitriptan release, six time points (1, 2, 3, 5, 12 and 22 hours) were collected during method development. Since drug release at the 1 hour time point was less than 30%, and more than 80% of drug was released at the 22 hour time point, time points 1, 3, 5, and 22 hours covered the early range with no dose dumping, the middle range and late stages of the release profile will be used in lot release or stability testing.

### 10. Agitation Study

The effect of dip rate on the drug release profile was evaluated during method development in order to obtain an optimal dip rate. Six samples from Lot 17010 were tested at 5, 30 and 60 dpm in 30 mL of 10 mM PBS release medium, at a temperature of 32° C. These samples displayed a release profile from 1 hour to 22 hours. Percent drug release was calculated based on the 1.9 mg zolmitriptan label claim. The effect of dip rate on mean percent drug release is listed in Table 10 and profiles are shown in FIG. 12. The drug release from the first two time points was similar at the 5 to 60 dip rates. The drug release was slightly faster at middle time points of 3 and 5 hours at 60 dpm. The drug released more than 85% at the 12 hour time points and more than 95% at the 22 hour time points for all three dip rates. The dip rate of 30 was selected to align with USP <724> dip rate requirement for transdermal system applying Apparatus 7.

TABLE 10
Effect of dipping rate on drug released profile (Sample size = 6

| | 17010 DPM 5 | | 17010 DPM 30 | | 17010 DPM 60 | |
|---|---|---|---|---|---|---|
| Time point | (n = 6) | RSD | (n = 6) | RSD | (n = 6) | RSD |
| 1 | 12% | 16% | 12% | 14% | 13% | 15% |
| 2 | 29% | 9% | 28% | 9% | 28% | 10% |
| 3 | 43% | 7% | 41% | 8% | 47% | 10% |
| 5 | 63% | 5% | 60% | 7% | 69% | 12% |
| 12 | 94% | 3% | 89% | 4% | 93% | 4% |
| 22 | 102% | 4% | 98% | 2% | 98% | 3% |

### 11. Bath Temperature Study

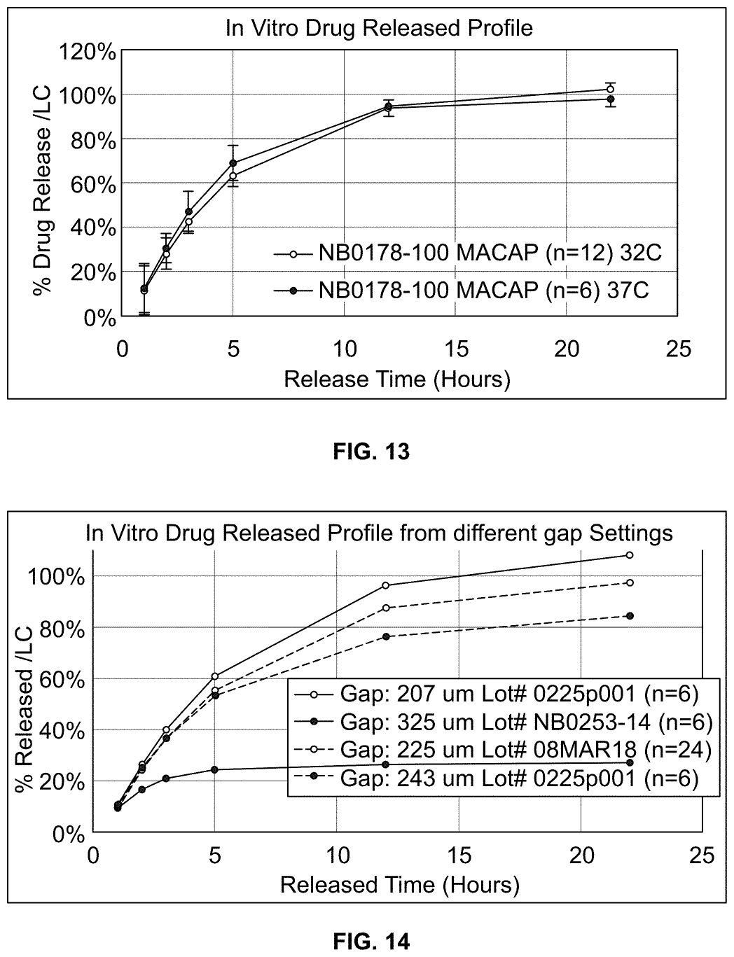

For in vitro drug release test for transdermal system, the solution containers were immersed in a water bath that maintained the temperature inside the containers at 32.0±0.5° C. per USP <724>. In vitro drug release profile of zolmitriptan from zolmitriptan patches at bath temperature of 37.0° C. was obtained for comparison purposes during method development. Six samples from lot NB0178-100 were tested at 32° C. and 37.0° C. separately. Drug release program and HPLC parameters were the same as listed in Table 2 and Table 1, except the bath temperature was modified. Table 11 and FIG. 13 are the drug release results in 10 mM PBS from two temperature conditions. The drug released at the first two time points are similar in both bath temperatures. The drug released slightly faster at middle time points of 3 and 5 hours in the bath with the higher temperature condition. The drug release was more than 90% at the 12 hour time points in both bath temperatures condition.

TABLE 11
Effect of Temperature on drug released profile

| Time | NB0178-100 | | NB0178-100 | |
|---|---|---|---|---|
| point | (n = 6) 32 C. | RSD | (n = 6) 37 C. | RSD |
| 1 | 12% | 11% | 13% | 11% |
| 2 | 28% | 7% | 31% | 7% |
| 3 | 43% | 6% | 47% | 9% |
| 5 | 63% | 5% | 69% | 8% |
| 12 | 94% | 4% | 95% | 3% |
| 22 | 103% | 3% | 98% | 4% |

### 12. Stability of Zolmitriptan in Release Medium

In order for the drug to have fully released from the patches, a complete release time of 22 hours was noted. Therefore, stability of zolmitriptan in the release medium was evaluated for up to 24 hours. The drug stability results in Table 12 indicated that zolmitriptan remained stable in 10 mM PBS release media for up to 24 hours at 32° C.

TABLE 12
Stability of Zolmitriptan in 10 mM PBS Release Media

| T0 Concentration | After 24 hours at 32° C. | % Drug Recovery After 24 |
|---|---|---|
| (μg/mL) | Concentration (μg/mL) | hours at 32° C. |
| 0.49 | 0.50 | 102% |
| 5.00 | 5.02 | 100% |
| 335.94 | 335.94 | 100% |

### 13. Discrimination Power Study

To evaluate the discriminatory power of the method, drug product with different process parameters settings were manufactured and tested. The percentage of drug release for each time point was calculated against the drug label claim (1.9 mg zolmitriptan).

Drug product manufactured from different process settings were tested to evaluate the drug release method discriminating power. Table 13 lists the various process parameters that were evaluated.

TABLE 13
Modified Process Parameters Groups

| | Passes | Pause | GAP | Arm & Drum |
|---|---|---|---|---|
| Group # | (No.) | (second) | (μm) | Speed (mm/sec) |
| Control (Nominal) | 5 | 2 | 225 | 70/70 |
| Low Gap | 5 | 3 | [symbol] | 70/70 |
| Highest Gap | 5 | 2 | [symbol] | 70/70 |
| Higher Arm | 5 | 2 | 225 | [symbol] |
| Low Pause | 5 | [symbol] | 225 | 70/70 |
| High Pause | 5 | [symbol] | 225 | 70/70 |
| Higher Pause | 5 | [symbol] | 225 | 70/70 |

Gap (arm setting) relates to the distance between the microneedles as they are secured by a vacuum chuck, and presented to the drug film reservoir in order to coat the needles. The nominal gap height is 225 um. Pause relates to the pause time between passes, which is nominally 2 seconds (each time the needles are presented to the reservoir to coat the needles) in order for the formulation to dry on the needles.

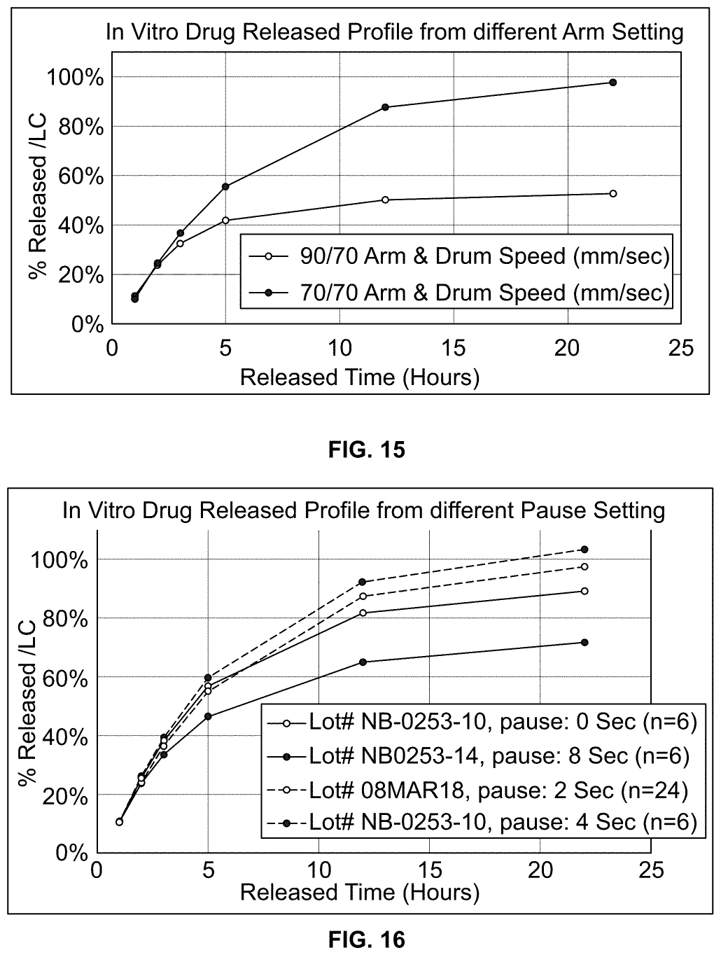

As shown in Table 14 to Table 16, and FIG. 14 to FIG. 16, the release profiles from: (1) patches processed with 325 um gap, (2) patches processed with higher arm settings, and (3) patches processed with higher pause times of 8 seconds are significantly different from patches manufactured under nominal conditions (as defined by control group in Table 13).

When compared to the drug release profiles from patches manufactured under nominal conditions, drug was released faster at the 22 hour time point from patches processed with a gap setting at 207 um, and released much slower from patches processed with a gap setting of 325 um or higher arm setting or pause time of 8 seconds. Based on the results observed, the method can discriminate between patches processed from different manufacturing process parameter settings.

TABLE 14
Drug released results from Patches Processed with Different Gap Settings

| Lot # | Mar. 8, 2018 | | 0225p001 | | 0225p001 | | NB0253-14 | |
|---|---|---|---|---|---|---|---|---|
| Gap | Gap: 225 μm (control) | | Gap: 207 μm | | Gap: 243 μm | | Gap: 325 μm | |
| Time point | (n = 24) | RSD | (n = 6) | RSD | (n = 6) | RSD | (n = 6) | RSD |
| 1 | 10% | 16% | 10% | 10% | 10% | 6% | 9% | 12% |
| 2 | 24% | 9% | 26% | 7% | 25% | 2% | 16% | 7% |
| 3 | 37% | 8% | 40% | 6% | 37% | 3% | 21% | 9% |
| 5 | 55% | 6% | 61% | 5% | 54% | 6% | 24% | 8% |
| 12 | 88% | 4% | 96% | 3% | 76% | 11% | 26% | 9% |
| 22 | 98% | 3% | 108% | 2% | 84% | 11% | 27% | 9% |

TABLE 15
Drug released results from Patches Process with Different
Arm & Drum Settings

| Arm & Drum | 70/70 Arm & Drum Speed | | 90/70 Arm & Drum | |
|---|---|---|---|---|
| Speed | (mm/sec)-control | | Speed (mm/sec) | |
| (mm/sec) | Lot# 08MAR18 | | Lot# NB0253-14 | |
| Time point | (n = 24) | RSD | (n = 6) | RSD |
| 1 | 10% | 16% | 11% | 6% |
| 2 | 24% | 9% | 24% | 4% |
| 3 | 37% | 8% | 32% | 4% |
| 5 | 55% | 6% | 42% | 3% |
| 12 | 88% | 4% | 50% | 3% |
| 22 | 98% | 3% | 52% | 2% |

TABLE 16
Drug released results from Patches Processed with Different Pauses Settings

| Lot # | Mar. 8, 2018 | | NB-0253-10 | | NB-0253-10 | | NB-0253-10 | |
|---|---|---|---|---|---|---|---|---|
| Gap | Pause: 2 seconds (control) | | Pause: 0 second | | Pause: 4 seconds | | Pause: 8 seconds | |
| Time point | (n = 24) | RSD | (n = 6) | RSD | (n = 6) | RSD | (n = 6) | RSD |
| 1 | 10% | 16% | 11% | 7% | 11% | 17% | 11% | 13% |
| 2 | 24% | 9% | 26% | 6% | 26% | 11% | 24% | 6% |
| 3 | 37% | 8% | 39% | 6% | 39% | 10% | 34% | 4% |
| 5 | 55% | 6% | 57% | 5% | 60% | 9% | 47% | 2% |
| 12 | 88% | 4% | 82% | 5% | 93% | 7% | 65% | 4% |
| 22 | 98% | 3% | 89% | 4% | 103% | 4% | 72% | 5% |

### Method

This method is employed to determine in vitro drug release of zolmitriptan from M207 patches by Apparatus 7 (Reciprocating Holder) per USP <724>. The patches with drug side out are attached to the custom-designed holders. The phase separator membrane is placed on top of each patch in the holder and firmly held by a clip.

The holders loaded with patches are then mounted to Apparatus 7 and dipped into the test tubes, which contains 30 mL of the phosphate buffered saline that is maintained at a constant temperature of 32±0.5° C. The samples are dipped into different tubes at a dip rate of 30 DPM according to the testing interval.

At the end of each dipping interval, the drug release media is transferred to HPLC vials and analyzed for drug content by reversed-phase HPLC. The amount of drug release at each sample collection time interval is added to the total amount of drug released from all the previous time intervals and the % Label Claim of cumulative drug release is reported for each patch. The method is outlined in TM-646 “In Vitro Drug Release of Zolmitriptan from Zolmitriptan Patch”.

Five calibration standard levels with a zolmitriptan concentration range of 0.5 μg/mL to 40 μg/mL are used to quantify the amount of drug released. System suitability is performed before every HPLC run. System suitability results are checked against the system suitability criteria stated in Table 17, below, before any samples are analyzed

TABLE 17
System Suitability Acceptance Criteria

| Description | | System Suitability Criteria | | |
|---|---|---|---|---|
| Mixture of Mobile A and | | No interference peak at 5% window of | | |
| Mobile B; Release Medium | | zolmitriptan retention time from first | | |
| (1XDPBS) | | injection of WS3. | | |
| System Suitability | | | | |
| (WS3, n = 5) | | | | |
| a) | zolmitriptan peak from | a) | Capacity Factor | k′ ≥ 0.9 |
| | the First injection of WS3 | | Tailing Factor (T): | 0.5 ≤ T 2.5 |
| b) | zolmitriptan peaks from | b) | % RSD of Retention | ≤2% |
| | all 5 injections of WS3 | | Time: | |
| | | | % RSD of Area | ≤2% |
| | | | Response: | |

| | |
|---|---|
| Zolmitriptan Calibration | R² ≥ 0.99 |
| Working Standards | |
| Zolmitriptan QC Standard | The % Difference of response factor |
| | (Peak Area/Concentration) between QC |
| | Standard and WS3 from calibration |
| | working standard should be no more |
| | than 2%. |

### Results and Discussion

Pre-validation studies were performed to ensure that the in vitro drug release method meets the method performance criteria for QC lot release testing. The results from pre-validation also provide information for setting of acceptance criteria for method validation work.

### 1. HPLC Method LOQ

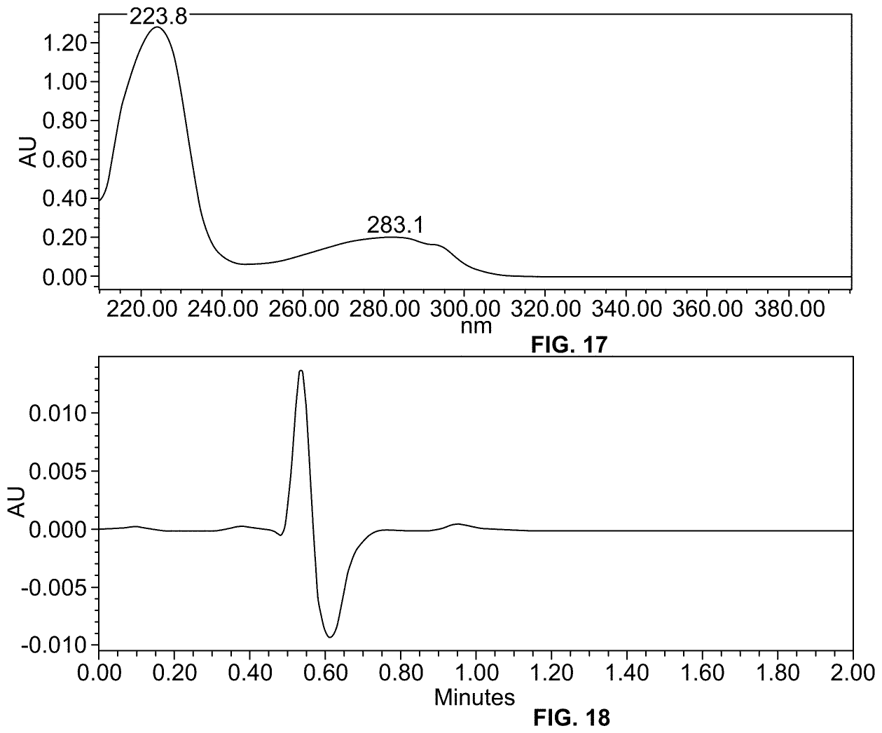

The amount of zolmitriptan eluted in the medium from each time interval is determined by RP-HPLC method. Zolmitriptan spectrum shows lambda max at wavelength 225 nm and 280 nm, with wavelength at 225 nm show significant higher absorption than 280 nm wavelength (see FIG. 17). To increase sensitivity of TM-600, UV wavelength at 225 nm was selected for in vitro drug release method. Based on LOQ study reported in TR-5186, “Method Development Report for Determination of Zolmitriptan Content in Cleaning Samples”, the estimated LOQ of TM-600 at wavelength 225 nm was 0.05 ug/ml. The HPLC method is sensitive enough to accurately quantify approximately 0.1% of drug released in the medium. The typical amount of zolmitriptan eluted from each time interval is within 5-20% of drug label claim.

### 2. Specificity

TM-600 method specificity has been verified during method validation and documented in VR-667, “Method validation of TM-600”. There is no interference from tartaric acid, water and extracted solution of uncoated array. Phosphate buffer is used as mobile phase component and does not interference with the zolmitriptan peak as indicated in chromatogram of Phosphate buffer (FIG. 18).

### 3. Repeatability

One analyst performed in vitro drug release testing on 12 patches from the same lot NB0178-100 to evaluate method repeatability. As shown in Table 18, for sample size of 12, the % RSD at the first time point (1 hour) is ≤15%; the % RSD at the remaining time points is ≤10%. Repeatability of method is acceptable.

TABLE 18
Repeatability Summary (Lot #NB0178-100
% Drug Released/Label Claim

| Sample ID | 1 hour | 2 hours | 3 hours | 5 hours | 12 hours | 22 hours |
|---|---|---|---|---|---|---|
| 1 | 11% | 28% | 43% | 65% | 95% | 103% |
| 2 | 12% | 28% | 43% | 64% | 92% | 100% |
| 3 | 11% | 28% | 43% | 64% | 96% | 104% |
| 4 | 13% | 31% | 46% | 68% | 99% | 107% |
| 5 | 12% | 28% | 43% | 64% | 93% | 103% |
| 6 | 8% | 22% | 34% | 52% | 85% | 98% |
| 7 | 12% | 28% | 43% | 64% | 94% | 102% |
| 8 | 13% | 31% | 46% | 68% | 100% | 107% |
| 9 | 11% | 27% | 41% | 61% | 92% | 101% |
| 10 | 14% | 31% | 45% | 65% | 96% | 104% |
| 11 | 11% | 27% | 42% | 62% | 92% | 101% |
| 12 | 10% | 26% | 39% | 60% | 91% | 101% |
| Average | 11% | 28% | 42% | 63% | 94% | 103% |
| SD | 1% | 2% | 3% | 4% | 4% | 3% |
| % RSD | 13% | 8% | 7% | 7% | 4% | 3% |

### 4. Intermediate Precision

Two analysts tested, individually, 6-7 patches from the same lot on different days to study intermediate precision for this method. The average values for the patches from each analyst and each day were summarized in Table 19. The difference between two average results from two analysts for all the time points is less than 10%. Although patches from same lot were tested by two analysts, higher variation is observed in analyst 2 drug released results and two units released less than 80% at 12 hour time point, which may be due to the difference of drug coating amounts on the tested patches as the patches were randomly picked from different bins process.

TABLE 19
Intermediate Precision Results Summary (Lot #NB0178-100
% Drug Released /Label Claim Performed By Analyst 1 (CL)

| Sample ID | 1 hour | 2 hours | 3 hours | 5 hours | 12 hours | 22 hours |
|---|---|---|---|---|---|---|
| 1 | 12% | 28% | 43% | 64% | 94% | 102% |
| 2 | 13% | 31% | 46% | 68% | 100% | 107% |
| 3 | 11% | 27% | 41% | 61% | 92% | 101% |
| 4 | 14% | 31% | 45% | 65% | 96% | 104% |
| 5 | 11% | 27% | 42% | 62% | 92% | 101% |
| 6 | 10% | 26% | 39% | 60% | 91% | 101% |
| Average | 12% | 28% | 43% | 63% | 94% | 103% |
| SD | 1% | 2% | 2% | 3% | 3% | 3% |
| %RSD | 11% | 7% | 6% | 5% | 4% | 3% |

% Drug Released /Label Claim Performed By Analyst 2 (HYZ)

| Sample ID | 1 hour | 2 hours | 3 hours | 5 hours | 12 hours | 22 hours |
|---|---|---|---|---|---|---|
| 1 | 11% | 26% | 41% | 61% | 87% | 102% |
| 2 | 11% | 28% | 44% | 65% | 91% | 103% |
| 3 | 8% | 22% | 34% | 51% | 74% | 87% |
| 4 | 10% | 27% | 42% | 64% | 93% | 107% |
| 5 | 11% | 28% | 44% | 66% | 93% | 104% |
| 6 | 8% | 21% | 33% | 49% | 73% | 90% |
| 7 | 10% | 26% | 41% | 62% | 89% | 101% |
| Average | 10% | 26% | 40% | 60% | 86% | 99% |
| SD | 1% | 3% | 5% | 7% | 9% | 8% |
| % RSD | 13% | 12% | 12% | 11% | 10% | 8% |
| % Difference | 2% | 2% | 3% | 3% | 8% | 4% |

### 5. Sample Solution Stability

Sample solutions containing the released drug after the drug release tests were stored at 2-8° C. for a few days and injected to check the stability of the solution. Six sample solutions from each time points were aliquoted from release test tube and stored in refrigerator at 2-8 C for 15 days before being analyzed by HPLC. The aged sample solutions drug amount results and percent recovery from fresh samples were listed in Table 20. Except for the drug amount of 0.1 mg in one sample solution eluted at the 22 hour time point, the drug recovery from fresh samples is within the range of 100-105%. For the solution concentration is close to 1×-5× of the quantitation limit, 85%-115% recovery from fresh sample is considered reasonable.

TABLE 20
Sample Solutions Stability Results

| Time Zero | Drug Amount (mg) | | | | | | |
|---|---|---|---|---|---|---|---|
| Time Point | #1 | #2 | #3 | #4 | #5 | #6 | Average |
| 1 hour | 0.21 | 0.2 | 0.19 | 0.24 | 0.22 | 0.2 | 0.21 |
| 2 hours | 0.33 | 0.34 | 0.32 | 0.32 | 0.33 | 0.31 | 0.33 |
| 3 hours | 0.28 | 0.3 | 0.28 | 0.26 | 0.28 | 0.26 | 0.28 |
| 5 hours | 0.41 | 0.48 | 0.43 | 0.36 | 0.42 | 0.37 | 0.41 |
| 12 hours | 0.59 | 0.85 | 0.75 | 0.45 | 0.63 | 0.51 | 0.63 |
| 22 hours | 0.15 | 0.27 | 0.24 | 0.09 | 0.17 | 0.12 | 0.17 |
| T = 15 days | Drug Amount (mg) | | | | | | |
| Time Point | #1 | #2 | #3 | #4 | #5 | #6 | Average |
| 1 hour | 0.22 | 0.20 | 0.20 | 0.25 | 0.23 | 0.21 | 0.22 |
| 2 hours | 0.33 | 0.35 | 0.33 | 0.32 | 0.33 | 0.32 | 0.33 |
| 3 hours | 0.29 | 0.31 | 0.30 | 0.26 | 0.29 | 0.26 | 0.29 |
| 5 hours | 0.41 | 0.48 | 0.44 | 0.36 | 0.42 | 0.38 | 0.42 |
| 12 hours | 0.60 | 0.83 | 0.76 | 0.46 | 0.64 | 0.51 | 0.63 |
| 22 hours | 0.15 | 0.28 | 0.25 | 0.10 | 0.17 | 0.12 | 0.18 |

% Recovery from Time Zero

| Time Point | #1 | #2 | #3 | #4 | #5 | #6 | Average |
|---|---|---|---|---|---|---|---|
| 1 hour | 105% | 100% | 105% | 104% | 105% | 105% | 104% |
| 2 hours | 100% | 103% | 103% | 100% | 100% | 103% | 102% |
| 3 hours | 104% | 103% | 107% | 100% | 104% | 100% | 103% |
| 5 hours | 100% | 100% | 102% | 100% | 100% | 103% | 101% |
| 12 hours | 102% | 98% | 101% | 102% | 102% | 100% | 101% |
| 22 hours | 100% | 104% | 104% | 111% | 100% | 100% | 103% |

### 6. Accuracy

Two levels of accuracy samples were prepared in triplicate by diluting the stock and working standard solutions with 10 mM PBS medium respectively (see Table 21 for sample preparation). Then 30 ml of the accuracy solutions were transferred to the drug release test tubes. The test tubes were placed into the release rate tester with a stent holder containing placebo patches. The accuracy solutions were incubated at 32° C. for one hour and then transferred to HPLC vials for drug concentration analysis. The accuracy results are listed in Table 22.

TABLE 21
Accuracy Samples Preparations Scheme

| | | | Spiked | Spiked | Volume | Total |
|---|---|---|---|---|---|---|
| | Target | Target | Standard | Standard | of Spiked | Final |
| | Release | Concentration | solution | Concentration | Standard | Volume |
| Sample ID | % LC | (μg/mL) | ID | (μg/mL) | (mL) | (mL) |
| A1-Low | ~1% | 0.5 | WS2 | 5 | 3 | 30 |
| Level | | | | | | |
| A2-Mid | ~5% | 3.0 | WS5 | 40 | 2 | 30 |
| Level | | | | | | |

TABLE 22
Summary of Accuracy Samples

| | Concentration | Theoretical | |
|---|---|---|---|
| | Found | Concentration | |
| Sample ID | (μg/mL) | (μg/mL) | % Recovery |
| A1 Low Prep 1 | 0.490 | 0.501 | 97.8 |
| A1 Low Prep 2 | 0.492 | | 98.2 |
| A1 Low Prep 3 | 0.497 | | 99.2 |
| | | Average | 98.4 |
| | | % RSD | 0.73 |
| A2 Mid Prep 1 | 2.641 | 2.651 | 99.6 |
| A2 Mid Prep 2 | 2.647 | | 99.8 |
| A2 Mid Prep 3 | 2.647 | | 99.8 |
| | | Average | 99.8 |
| | | % RSD | 0.13 |

### 7. Linearity and Range

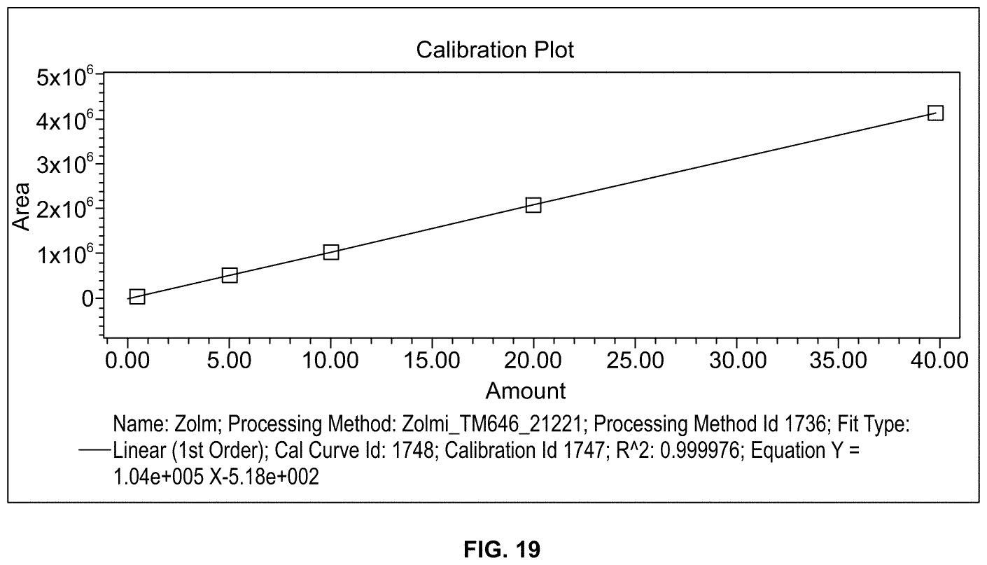

A series of zolmitriptan USP standard solutions was prepared and injected, by diluting the drug with 10 mM PBS: 0.5, 5, 10, 20, and 40 μg/ml. A calibration curve was plotted by response area verse concentration (FIG. 19). The trend line is linear. The square of regression coefficient (r²) is 1.000. The drug content in 10 mM PBS in this method is linear through the range from 0.5 μg/ml to 40 μg/ml as demonstrated in FIG. 19 and Table 23.

TABLE 23
Linearity Result Summary

| | Zolmitriptan | Zolmitriptan | |
|---|---|---|---|
| | Theoretical | Measured | |
| Sample | Concentration | Concentration | |
| Name | (μg/mL) | (μg/mL) | % deviation |
| WS1 | 0.501 | 0.495 | −1.2 |
| WS2 | 5.005 | 5.021 | 0.3 |
| WS3 | 10.000 | 9.911 | −0.9 |
| WS4 | 19.959 | 20.077 | 0.6 |
| WS5 | 39.761 | 39.722 | −0.1 |

| | |
|---|---|
| R-Square | 1.000 |
| Slope | 1.04 e+005 |
| y-Intercept | −5.18 e+002 |

### CONCLUSION

The test method described above (TM-646) for the determination of in vitro drug release profile of zolmitriptan from M207 patches was developed. One to twelve hours, multiple time point in vitro release profile of zolmitriptan from M207 patches with less than 30% drug released at the first time point, and more than 80% drug eluted at the last time point was achieved.

In vitro drug release profiles generated from patches processed from variable critical manufacturing parameters show a significant difference in the release profile from patches manufactured under nominal conditions.

The method has enough discrimination power to differentiate between patches processed with different manufacturing process parameter settings. A pre-validation study was performed to determine the method linearity, accuracy, repeatability, intermediate precision, and robustness. Results from method development demonstrated the test method TM-646 is suitable for the intended use.

The embodiments of the present disclosure described above are intended to be merely exemplary; numerous variations and modifications will be apparent to those skilled in the art. All such variations and modifications are intended to be within the scope of the present invention as defined in any appended claim.

We claim:

1. A method for determining the in vitro dissolution profile of a bioactive agent from a microneedle patch, comprising the steps of:
a. providing a microneedle patch comprising a bioactive agent;
b. affixing the patch to an outer surface of a sample holder wherein the microneedles are oriented outward from the outer surface;
c. applying a membrane over the microneedles and the patch;
d. immersing the sample holder having the patch and membrane in a dissolution medium;
e. taking at least 2 samples of the dissolution medium at different time points;
f. determining the concentration of the bioactive agent in the samples; and
g. calculating the rate of dissolution of the bioactive agent from the patch.

2. The method of claim 1 further comprising attaching a clip over the membrane and sample holder.

3. The method of claim 2 wherein the clip has an opening.

4. The method of claim 3 wherein the opening is a shape selected from the group consisting of rectangular, square, oblong, circular, parallelogram, pentagon, octagon, hexagon, rhombus, oval, and triangular.

5. The method of claim 3 wherein the opening is a size of about 10 cm², or 9 cm², or 8 cm², or 7 cm², or 6 cm², or 5 cm², or 4 cm², or 3 cm², or 2 cm², or 1 cm².

6. The method of claim 1 wherein the dissolution medium has a volume of about 50 mL to about 200 mL.

7. The method of claim 1 wherein the dissolution medium has a volume of about 900 mL.

8. The method of claim 1 wherein the dissolution medium has a temperature of about 32 degrees.

9. The method of claim 1 wherein less than about 30% of the drug is released at a first time point, and more than about 80% of the drug is released at a last time point.

10. The method of claim 1 wherein less than about 20%-40% of the drug is released at a first time point, and more than about 60%-90% of the drug is released at a second time point.

11. The method of claim 1 wherein the method yields data with reasonable variability having a relative standard deviation (RSD) from a first time point being about <20% and a RSD from other time points being about <10% for a sample size of 12.
